(19) United States
(12) Patent Application Publication
(10) Pub. No.: US 2024/0374934 A1
(43) Pub. Date: Nov. 14, 2024

(54) TISSUE TREATMENT CATHETER HAVING PORT BRACE

(71) Applicant: Otsuka Medical Devices Co., Ltd., Tokyo (JP)

(72) Inventors: James D. Mazzone, San Jose, CA (US); Desmond CHEUNG, San Jose, CA (US); John R. Stetak, III, San Jose, CA (US)

(21) Appl. No.: 18/659,628

(22) Filed: May 9, 2024

Related U.S. Application Data
(60) Provisional application No. 63/501,347, filed on May 10, 2023.
(60) Provisional application No. 63/586,639, filed on Sep. 29, 2023.

(51) Int. Cl. A61N 7/02; A61M 25/00; A61M 25/10; A61N 7/00

(52) U.S. Cl. CPC A61N 7/02; A61M 25/0013; A61M 25/0023; A61M 25/1034; A61B 2217/007; A61M 25/10; A61N 2007/0026; A61N 2007/003

(57) ABSTRACT

A tissue treatment catheter includes a catheter shaft having a guidewire lumen and a guidewire port extending through an outer shaft wall between the guidewire lumen and a surrounding environment. The tissue treatment catheter includes a port brace disposed in the guidewire lumen. The port brace includes a proximal brace section in the guidewire lumen proximal to the guidewire port. The port brace includes a distal brace section in the guidewire lumen distal to the guidewire port. The proximal brace section is stiffer than the distal brace section. Other embodiments are also described and claimed.

This application claims the benefit of priority of U.S. Provisional Patent Application No. 63/501,347, filed on May 10, 2023, titled “Tissue Treatment Catheter Having Port Brace,” and U.S. Provisional Patent Application No. 63/586,639, filed on Sep. 29, 2023, titled “Tissue Treatment Catheter Having Port Brace,” both of which are incorporated herein by reference in their entirety to provide continuity of disclosure.

### BACKGROUND

### Field

This application relates generally to minimally-invasive apparatuses, systems, and methods that provide energy delivery to a targeted anatomical location of a subject, and more specifically, to apparatuses, systems, and methods for the treatment of tissue, such as nerve tissue.

### BACKGROUND INFORMATION

High blood pressure, also known as hypertension, commonly affects adults. Left untreated, hypertension can result in renal disease, arrhythmias, and heart failure. In recent years, the treatment of hypertension has focused on interventional approaches to inactivate the renal nerves surrounding a renal artery. Autonomic nerves tend to follow blood vessels to the organs that they innervate. Intraluminal devices, such as catheters, may reach specific structures, such as the renal nerves, which are proximate to the lumens in which the catheters travel. Accordingly, catheter-based systems can deliver energy from within the lumens to inactivate the renal nerves in the vessel walls.

An ultrasound transducer can be mounted at a distal end of catheter, and an unfocused ultrasound energy can heat tissue adjacent to a body lumen within which the catheter (and the transducer) is disposed. Such unfocused ultrasound energy may, for example, denervate target nerves surrounding the body lumen, without damaging non-target tissue such as the inner lining of the body lumen or unintended organs outside of the body lumen. The unfocused ultrasound energy system may also include a balloon mounted at the distal end of the catheter around the ultrasound transducer. A cooling fluid can be circulated through the balloon to cool the body lumen during ultrasound energy delivery. Such a design enables creation of one or more ablation zones sufficient to achieve long-term nerve inactivation at different locations around the circumference of the blood vessel.

### SUMMARY

The present invention is defined in the independent claims. Further embodiments of the invention are defined in the dependent claims.

A tissue treatment catheter is provided herein. A tissue treatment catheter includes a catheter shaft having a guidewire lumen and a guidewire port extending through an outer shaft wall between the guidewire lumen and a surrounding environment. The tissue treatment catheter includes a port brace disposed in the guidewire lumen. The port brace includes a proximal brace section in the guidewire lumen proximal to the guidewire port. The port brace includes a distal brace section in the guidewire lumen distal to the guidewire port. The proximal brace section is stiffer than the distal brace section.

A tissue treatment catheter is provided herein. The tissue treatment catheter includes a catheter shaft having a fluid lumen, a cable lumen, a guidewire lumen, and a guidewire port. The guidewire port extends through an outer shaft wall between the guidewire lumen and a surrounding environment. The tissue treatment catheter includes a balloon mounted on the catheter shaft. The balloon has an interior in fluid communication with the fluid lumen. The tissue treatment catheter includes a port brace located in the fluid lumen or the cable lumen. The port brace is aligned with the guidewire port. The port brace is stiffer than the outer shaft wall at the guidewire port.

A tissue treatment catheter is provided herein. The tissue treatment catheter includes a catheter shaft having a fluid lumen, a cable lumen, a guidewire lumen, and a guidewire port. The guidewire port extends through an outer shaft wall between the guidewire lumen and a surrounding environment. The tissue treatment catheter includes a balloon mounted on the catheter shaft. The balloon has an interior in fluid communication with the fluid lumen. The tissue treatment catheter includes a port brace mounted on the outer shaft wall. The port brace is aligned with the guidewire port. The port brace is stiffer than the outer shaft wall at the guidewire port.

A tissue treatment catheter is provided herein. The tissue treatment catheter includes a catheter shaft having a guidewire lumen and a guidewire port. The guidewire port extends through an outer shaft wall between the guidewire lumen and a surrounding environment. The tissue treatment catheter includes a port brace having a brace lumen. The brace lumen is coaxial with the guidewire lumen. The port brace includes a proximal brace section proximal to the guidewire port. The port brace includes a distal brace section distal to the guidewire port.

A tissue treatment catheter is provided herein. The tissue treatment catheter includes a catheter shaft having an outer shaft wall extending around a stylet lumen coaxially aligned with a guidewire lumen. The catheter shaft includes a port edge defining a guidewire port extending through the outer shaft wall into the guidewire lumen. The outer shaft wall has a collapsed section tapering outward from the port edge longitudinally between the stylet lumen and the guidewire lumen. The tissue treatment catheter includes a stylet disposed in the stylet lumen.

A method of manufacturing a tissue treatment catheter is provided herein. The method includes forming a guidewire port through an outer shaft wall of a catheter shaft. The outer shaft wall extends around a stylet lumen coaxially aligned with a guidewire lumen. The guidewire port has a port edge. The method includes collapsing the outer shaft wall to form a collapsed section tapering outward from the port edge longitudinally between the stylet lumen and the guidewire lumen. The method includes inserting a stylet into the stylet lumen.

The above summary does not include an exhaustive list of all aspects of the present invention. It is contemplated that the invention includes all systems and methods that can be practiced from all suitable combinations of the various aspects summarized above, as well as those disclosed in the Detailed Description below and particularly pointed out in the claims filed with the application. Such combinations have particular advantages not specifically recited in the above summary.

### BRIEF DESCRIPTION OF THE DRAWINGS

The various features of the present disclosure and the manner of attaining them will be described in greater detail with reference to the following description, claims, and drawings, wherein reference numerals are reused, where appropriate, to indicate a correspondence between the referenced items, and wherein:

FIG. 1 is a perspective view of a portion of a tissue treatment system, in accordance with an embodiment.

FIG. 2 is a perspective view of a tissue treatment catheter, in accordance with an embodiment.

FIG. 3 is a perspective view of a tissue treatment catheter delivered into a body lumen, in accordance with an embodiment.

FIG. 4 is a top view of a guidewire port of a tissue treatment catheter, in accordance with an embodiment.

FIG. 5 is a partial cross-sectional view of a tissue treatment catheter, in accordance with an embodiment.

FIG. 6 is a side view of a port brace, in accordance with an embodiment.

FIG. 7 is a perspective view of a port brace, in accordance with an embodiment.

FIG. 8 is a side view of a brace tube of a port brace, in accordance with an embodiment.

FIG. 9 is a side view of a brace mandrel of a port brace, in accordance with an embodiment.

FIGS. 10-13 are cross-sectional views, taken at several locations along a longitudinal axis of a tissue treatment catheter, in accordance with an embodiment.

FIG. 14 is a cross-sectional view of a tissue treatment catheter having a port brace outside of a non-guidewire lumen, in accordance with an embodiment.

FIG. 15 is a perspective view of a port brace, in accordance with an embodiment.

FIG. 16 is a side view of a brace tube of a port brace, in accordance with an embodiment.

FIG. 17 is a side view of a brace tube transition, in accordance with an embodiment.

FIG. 18 is a partial cross-sectional view of a tissue treatment catheter, in accordance with an embodiment.

FIG. 19 is a perspective view of a port brace, in accordance with an embodiment.

FIG. 20 is a top view of a brace tube of a port brace, in accordance with an embodiment.

FIGS. 21-23 are cross-sectional views of a brace tube, taken about the section lines of FIG. 20, in accordance with an embodiment.

FIG. 24 is a perspective view of a guidewire port of a tissue treatment catheter, in accordance with an embodiment.

FIG. 25 is a top view of a guidewire port of a tissue treatment catheter, in accordance with an embodiment.

FIG. 26 is a cross-sectional view of a guidewire port of a tissue treatment catheter, in accordance with an embodiment.

FIG. 27 is a top view of a tissue treatment catheter, in accordance with an embodiment.

FIG. 28 is a cross-sectional view, taken about line A-A of FIG. 27, of a distal wall portion of a tissue treatment catheter, in accordance with an embodiment.

FIG. 29 is a cross-sectional view, taken about line B-B of FIG. 27, of a guidewire port of a tissue treatment catheter, in accordance with an embodiment.

FIG. 30 is a cross-sectional view, taken about line C-C of FIG. 27, of a ramp of a tissue treatment catheter, in accordance with an embodiment.

FIG. 31 is a cross-sectional view, taken about line D-D of FIG. 27, of a proximal wall portion of a tissue treatment catheter, in accordance with an embodiment.

FIG. 32 is a flowchart of a method of manufacturing a tissue treatment catheter, in accordance with an embodiment.

### DETAILED DESCRIPTION

Systems that use unfocused ultrasound energy to treat tissue, and methods of using the same, are provided herein. In certain embodiments, acoustic-based tissue treatment transducers, apparatuses, systems, and portions thereof, are provided. The systems may be catheter-based. The systems may be delivered intraluminally (e.g., intravascularly) so as to place a transducer within a target anatomical region of the subject, for example, within a suitable body lumen such as a blood vessel. When properly positioned within the target anatomical region, the transducer can be activated to deliver unfocused ultrasonic energy radially outward so as to suitably heat, and thus treat, tissue within the target anatomical region. The transducer or piezoelectric material can be activated, e.g., energized, at a frequency, duration, and energy level suitable for treating the ablation target, e.g., the targeted tissue. In one non-limiting example, unfocused ultrasonic energy generated by the transducer or piezoelectric material or radio frequency (RF) energy transmitted by the electrodes may target select nerve tissue of the subject, and may heat such tissue in such a manner as to neuromodulate (e.g., fully, or partially ablate, necrose, or stimulate) the nerve tissue.

Neuromodulating renal nerves may be used to treat various conditions, e.g., hypertension, chronic kidney disease, atrial fibrillation, autonomic nervous system for use in treating a variety of medical conditions, arrhythmia, heart failure, end stage renal disease, myocardial infarction, anxiety, contrast nephropathy, diabetes, metabolic disorder, and insulin resistance, etc. It should be appreciated, however, that the balloon catheters suitably may be used to treat other nerves and conditions, e.g., sympathetic nerves of the hepatic plexus within a hepatic artery responsible for blood glucose levels important to treating diabetes, or any suitable tissue, e.g., heart tissue triggering an abnormal heart rhythm, and is not limited to use in treating (e.g., neuromodulating) renal nerve tissue. In another example, a tissue treatment catheter is used to ablate sympathetic nerves of the renal arteries and a hepatic artery to treat diabetes or other metabolic disorders. In certain embodiments, the tissue treatment catheters are used to treat an autoimmune and/or inflammatory condition, such as rheumatoid arthritis, sepsis, Crohn's disease, ulcerative colitis, and/or gastrointestinal motility disorders by neuromodulating sympathetic nerves within one or more of a splenic artery, celiac trunk, superior or inferior mesenteric artery. In certain embodiments, the tissue treatment catheter is used to ablate nerve fibers in the celiac ganglion and/or renal arteries to treat hypertension. In certain embodiments, the transducers are used to treat pain, such as pain associated with pancreatic cancer, by, e.g., neuromodulating nerves that innervate the pancreas. Ultrasound or RF energy may also be used to ablate nerves of both the pulmonary vein and the renal arteries to treat atrial fibrillation. In still other examples, ultrasound or RF energy may additionally or alternatively be used to ablate nerves innervating a carotid body in order to treat hypertension and/or chronic kidney disease.

Existing tissue treatment catheters track over a guidewire to access a target anatomical region. Such tissue treatment catheters may include a guidewire lumen having a guidewire port. The guidewire port may be a rapid exchange port, which is typically a slot in a catheter wall midway between a distal end and a proximal end of the catheter. A guidewire can exit the guidewire lumen through the guidewire port. The guidewire port removes material from the catheter wall and, thus, creates a kink point. More particularly, the catheter wall surrounding the rapid exchange slot has an increased likelihood of kinking or buckling under the axial loads applied during device delivery. Accordingly, the weakened wall strength inherent in existing rapid exchange port configurations can lead to reduced pushability, given that excessive pushing forces can cause catheter buckling, and may result in an inability to access the target anatomical region.

As described below, embodiments can include a tissue treatment catheter and methods of manufacturing the tissue treatment catheter. The tissue treatment catheter may be an ultrasound-based tissue treatment catheter, used to deliver unfocused ultrasonic energy radially outwardly to treat tissue within a target anatomical region, such as the renal nerves within a renal artery. Alternatively, the tissue treatment system may be used in other applications, such as to treat sympathetic nerves of the hepatic plexus within a hepatic artery. Thus, reference to the system as being a renal denervation system, or being used in treating, e.g., neuromodulating, renal nerve tissue is not limiting.

In various embodiments, description is made with reference to the figures. However, certain embodiments may be practiced without one or more of these specific details, or in combination with other known methods and configurations. In the following description, numerous specific details are set forth, such as specific configurations, dimensions, and processes, in order to provide a thorough understanding of the embodiments. In other instances, well-known processes and manufacturing techniques have not been described in particular detail in order to not unnecessarily obscure the description. Reference throughout this specification to “one embodiment,” “an embodiment,” or the like, means that a particular feature, structure, configuration, or characteristic described is included in at least one embodiment. Thus, the appearance of the phrase “one embodiment,” “an embodiment,” or the like, in various places throughout this specification are not necessarily referring to the same embodiment. Furthermore, the particular features, structures, configurations, or characteristics may be combined in any suitable manner in one or more embodiments.

The use of relative terms throughout the description may denote a relative position or direction. For example, “distal” may indicate a first direction. Similarly, “proximal” may indicate a second direction, opposite to the first direction. Such terms are provided to establish relative frames of reference, however, and are not intended to limit the use or orientation of a tissue treatment catheter to a specific configuration described in the various embodiments below.

In an aspect, a tissue treatment catheter includes a rapid exchange guidewire port configuration that reduces a likelihood of kinking or buckling of a catheter wall surrounding the port. The tissue treatment catheter includes a port brace disposed in a guidewire lumen having the guidewire port. For example, a brace lumen of the port brace can be coaxially aligned with the guidewire lumen. The port brace reinforces the catheter wall around the guidewire port and, thus, increases the axial loading required to cause catheter buckling. More particularly, the tissue treatment catheter can have greater pushability and be less susceptible to buckling. The port brace may also have a variable stiffness that decreases in a distal direction. The variable stiffness can allow the tissue treatment catheter to track through tortuous vasculature by transitioning the port brace stiffness into the catheter wall stiffness. Accordingly, the port brace can enhance pushability and steerability of the tissue treatment catheter.

In an aspect, the rapid exchange guidewire port configuration that reduces the likelihood of kinking or buckling of the catheter wall includes a collapsed section. More particularly, the catheter wall can be collapsed, reflowed, or otherwise deformed from a cylindrical shape to form a ramp that extends outward from a guidewire port between a guidewire lumen and a stylet lumen. A stylet can be disposed in the stylet lumen to support the catheter shaft against buckling and provide pushability. The ramp can guide a guidewire from the guidewire lumen to a surrounding environment, and the tissue treatment catheter can be delivered over the guidewire to tortuous and small anatomies.

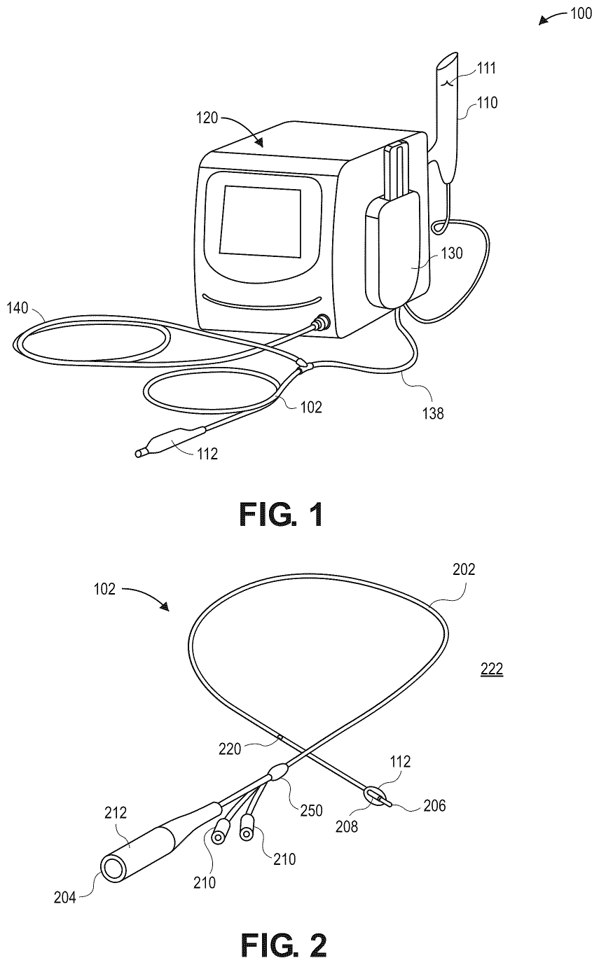

Referring to FIG. 1, a perspective view of a portion of a tissue treatment system is shown in accordance with an embodiment. A tissue treatment system 100 is shown as including a tissue treatment catheter 102 connected to a controller 120 by a connection cable 140. In certain embodiments, the tissue treatment catheter 102 includes an ultrasound transducer (FIG. 2) within a balloon 112. The tissue treatment system 100 can include a fluid reservoir 110 to store an inflation fluid 111. The inflation fluid 111 may be a cooling fluid. The tissue treatment system 100 can include, e.g., integrated within the controller 120, a fluid transfer unit 130 to transfer or move the inflation fluid 111 into and out of the balloon 112. More particularly, the fluid transfer unit 130 of the tissue treatment system 100 may deliver the inflation fluid 111 at an inflation pressure to the balloon 112, as described below. The tissue treatment system 100 may also include a cooling unit, e.g., integrated within the controller 120, to cool the inflation fluid 111. Accordingly, the inflation fluid 111 can be delivered to the balloon 112 by the fluid transfer unit 130 at a temperature below ambient temperature. In an embodiment, the tissue treatment system 100 includes an energy delivery unit configured to control activation, e.g., energize, the ultrasound transducer to deliver energy to the target anatomy.

In the embodiment shown in FIG. 1, the controller 120 is connected to the tissue treatment catheter 102 through an inflation tubing 138 for fluid transfer, and the connection cable 140 for electrical communication. In certain embodiments, the controller 120 interfaces with the fluid transfer unit 130 to provide the inflation fluid 111 to the tissue treatment catheter 102 for selectively inflating and deflating the balloon 112. The balloon 112 can be made from a biocompatible material. For example, the balloon 112 may be formed from a biocompatible elastomeric material. Examples of balloon materials that can be used to form the balloon 112 include, but are not limited to, nylon, a polyimide film, a thermoplastic elastomer (such as those marked under the trademark PEBAX™), a medical-grade thermoplastic polyurethane elastomer (such as Pellethane®, Isothane®, or other suitable polymers or any combination thereof), a silicone material, etc. The balloon material may be configured to transmit acoustic energy. More particularly, a wall of the balloon can be transparent to, and pass, acoustic energy, e.g., from an inner balloon surface to an outer balloon surface.

Referring to FIG. 2, a perspective view of a tissue treatment catheter is shown in accordance with an embodiment. The catheter-based intraluminal tissue treatment catheter 102 of the tissue treatment system 100 can include a catheter shaft 202 having an elongated body extending from a proximal catheter end 204 to a distal catheter end 206. The balloon 112 may be mounted on the catheter shaft 202, e.g., at the distal catheter end 206. One or more energy transducers, such as an ultrasound transducer 208, may be mounted on the catheter shaft 202. For example, the ultrasound transducer 208 may be positioned on the catheter shaft 202 within the balloon 112.

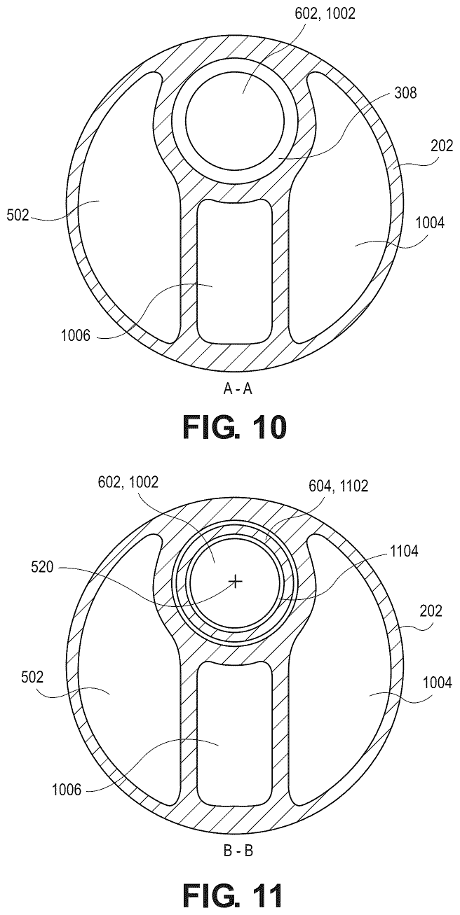

The catheter shaft 202 can include one or more lumens (e.g., FIG. 10), such as: fluid lumen(s) to deliver an inflation/cooling fluid to the balloon 112, cable lumen(s) to provide electrical cable passageways containing electrical cables to deliver energy to the transducer 208, guidewire lumens for exchanging guidewires, etc. The lumen(s) may be connected to corresponding connectors and/or terminal features, such as at the proximal catheter end 204. For example, the fluid lumens may connect to one or more fluid ports 210, which receive inflation/cooling fluid from the fluid transfer unit 130 of the tissue treatment system 100. Similarly, the electrical cables can connect to an external connector 212, which receives energy from a generator of the tissue treatment system 100 through the connection cable 140. In an embodiment, a terminal feature of the guidewire lumen is a guidewire port 220, located along the catheter shaft 202 between the distal catheter end 206 and the proximal catheter end 204. The guidewire port 220 allows a guidewire to pass between the guidewire lumen and a surrounding environment 222, as described below.

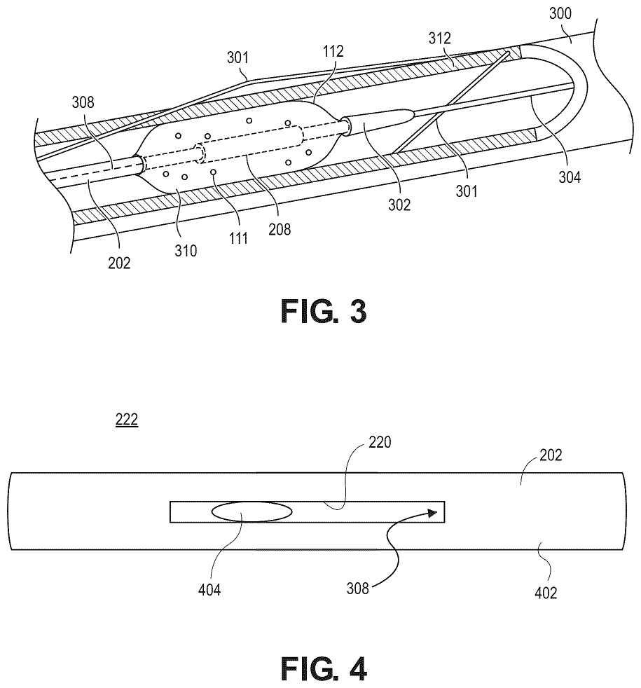

Referring to FIG. 3, a perspective view of a tissue treatment catheter delivered into a body lumen is shown in accordance with an embodiment. A distal portion of the tissue treatment catheter 102 may be inserted into a body lumen of a subject. The body lumen may be a vessel 300, e.g., a blood vessel such as a renal artery, which has a plurality of nerves 301. The vessel 300 can be a target vessel of an ablation procedure. More particularly, the nerves 301 can be an ablation target. The nerves 301 can surround the body lumen. For example, the nerves 301 may run in and around the blood vessel 300.

The distal portion of the tissue treatment catheter 102 may include the ultrasound transducer 208, the balloon 112 filled with the inflation fluid 111, the catheter shaft 202, and/or a guidewire support tip 302 configured to receive a guidewire 304. More particularly, the guidewire 304 can enter a guidewire lumen 308 through the guidewire support tip 302 and extend proximally through the catheter shaft 202 in the guidewire lumen to exit the catheter shaft 202 through the guidewire port 220 into the surrounding environment 222. The tissue treatment catheter 102 may therefore be tracked over the guidewire into the body lumen. The transducer 208 may be disposed partially or completely within the balloon 112. More particularly, the balloon 112 can contain the transducer 208 within an interior 310 of the balloon.

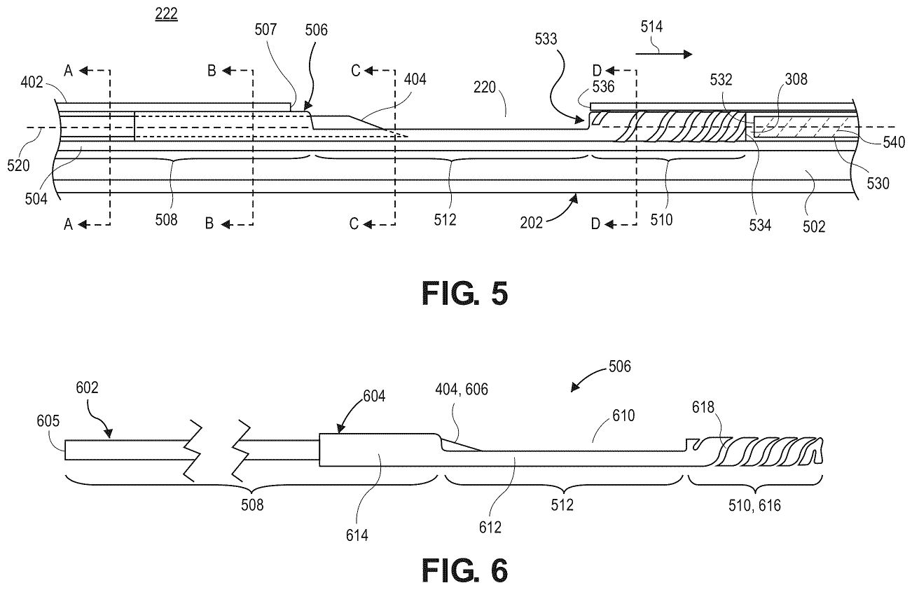

In an embodiment, the balloon 112 is adapted to inflate within a target anatomy, e.g., the vessel 300. More particularly, the balloon 112 may be inflated with the inflation fluid 111. The inflation fluid 111 can include a liquid. The liquid may have a relatively high, as compared to gases, thermal capacity. For example, the liquid may include water, dextrose, or saline, and have a corresponding heat capacity. The interior 310 of the balloon 112 may be in fluid communication with a fluid lumen (FIG. 5) extending through the catheter shaft 202. When the inflation fluid 111 is delivered through the fluid lumen and transferred into the interior 310 of the balloon 112, the balloon can inflate into contact with a vessel wall 312 of the vessel 300. The vessel wall 312, and/or the nerves 301 extending within and around the vessel wall, can be an ablation target.

In certain embodiments, the energy transducer 208 can be adapted to deliver ablation energy, e.g., ultrasound energy, to the target anatomy during a medical procedure, e.g., a renal denervation procedure. For example, the transducer 208 may be configured to emit acoustic energy in one or more, e.g., several, energy lobes toward the target anatomy. More particularly, the transducer 208 may be used to output acoustic energy to ablate the ablation target. During energy emission, the inflation fluid 111 can be circulated within the interior 310, around the transducer 208. Accordingly, the inflation fluid 111 can act as a heat sink to absorb heat generated by the ultrasound transducer 208 and/or delivered to the ablation target from the ultrasound transducer.

In certain embodiments, e.g., suitable for renal denervation, the balloon 112 is inflated while inserted in the body lumen of the patient during a procedure at a working pressure of about 10 to about 30 psi using the inflation fluid 111. The balloon 112 may be or include a compliant, semi-compliant, or non-compliant medical balloon. The balloon 112 is sized for insertion in the body lumen and, in the case of insertion into the renal artery, for example, the balloon 112 may be selected from available sizes including outer diameters of 3.5, 4.2, 5, 6, 7, or 8 mm, but not limited thereto. When activated, the transducer 208 can deliver the acoustic energy to the vessel wall 312 of the target vessel 300. The delivered energy can ablate and raise a temperature of the ablation target. The cooling fluid within the balloon 112, however, can be static and absorb heat to passively cool the ablation target and protect the target tissue and the transducer 208.

Referring to FIG. 4, a top view of a guidewire port of a tissue treatment catheter is shown in accordance with an embodiment. The catheter shaft 202 can have an outer shaft wall 402 facing radially outward toward the surrounding environment 222. The outer shaft wall 402 can surround the internal lumens of the catheter shaft 202, such as the guidewire lumen 308. In an embodiment, the guidewire port 220 of the catheter shaft 202 extends through the outer shaft wall 402 between the guidewire lumen 308 and the surrounding environment 222. More particularly, the guidewire port 220 can be an opening in the outer shaft wall 402 to allow the passage of the guidewire 304 from the guidewire lumen 308 to the surrounding environment 222.

The guidewire port 220 can have a perimeter or a port edge defining the opening from the catheter shaft 202 to the surrounding environment 222. The perimeter may, for example, be rectangular as shown in FIG. 4. Alternatively, the perimeter may be elliptical or other-shaped. The guidewire port 220 may be formed using a skiving process in which a portion of the outer shaft wall 402 is removed over the guidewire lumen 308 while portions not over the guidewire lumen 308 (such as portions over the fluid lumen(s)) are kept intact. The guidewire lumen 308 may have a diameter in a range of about 0.015-0.025 inch, e.g., about 0.021 inch. Accordingly, the guidewire lumen 308 is a compact area for inclusion of a port brace, as described below.

The tissue treatment catheter 102 may include a guidewire ramp 404 aligned with the guidewire port 220. The guidewire ramp 404 can be an angled and/or contoured surface shaped to direct the guidewire 304 from a primarily longitudinal direction extending through the guidewire lumen 308 to a radially outward direction through the guidewire port 220. More particularly, the guidewire ramp 404 can deflect and redirect the guidewire 304 to exit the catheter shaft 202.

The guidewire port 220 and the guidewire ramp 404 configurations seen in the top view may appear to be formed using known methods of reforming and or filling the catheter lumens while a mandrel is located within the guidewire lumen 308. Molding the guidewire port 220 and guidewire ramp 404 in such a manner may not, however, provide sufficient support around the guidewire port 220 to avoid catheter buckling. Furthermore, such polymer reflowing techniques used to form the guidewire port 220 and the guidewire ramp 404 may not provide a variable stiffness in the longitudinal direction within the port region. Accordingly, pushability and steerability of catheter shafts formed using reflowing techniques, other than those described below, may provide unacceptable performance. As described below, in an embodiment, the tissue treatment catheter 102 includes a port brace to enhance pushability and steerability of the tissue treatment catheter 102.

Referring to FIG. 5, a partial cross-sectional view of a tissue treatment catheter is shown in accordance with an embodiment. The catheter shaft 202 includes the guidewire lumen 308 and a fluid lumen 502 extending longitudinally through the catheter shaft 202, radially inward from the outer shaft wall 402. The shaft lumens such as the guidewire lumen 308 and the fluid lumen 502, may be separated from each other within the catheter shaft 202 by one or more lumen walls 504. For example, the entire catheter shaft wall structure, including the outer shaft wall 402 and the lumen walls 504, may be formed as a single shaft member in a polymer extrusion process. Accordingly, the catheter shaft wall structure can have a same stiffness along its length from a proximal end of the catheter shaft 202 to a distal end of the catheter shaft 202.

In an embodiment, a stiffness of the tissue treatment catheter 102 in the guidewire port 220 region is altered by a port brace 506. The port brace 506 can be disposed in the guidewire lumen 308. The port brace 506 can span a length of the guidewire port 220. For example, the guidewire port 220 can be defined by the port edge extending around the port, and the port edge can have a proximal port edge 507 at a proximal end of the guidewire port 220 and a distal port edge 536 at a distal end of the guidewire port. The port brace 506 can include a proximal brace section 508 located in the guidewire lumen 308 proximal to the guidewire port 220, e.g., proximal to the proximal port edge 507, and a distal brace section 510 located in the guidewire lumen 308 distal to the guidewire port 220, e.g., distal to the distal port edge 536. The distal and proximal brace sections may be bridged by a medial brace section 512 extending over the length of the guidewire port 220. The medial brace section 512 can align with the guidewire port 220. More particularly, the medial brace section 512 may radially appose the guidewire port 220. Accordingly, the port brace 506 can extend in a distal direction 514 within the guidewire lumen 308 through sections of the catheter shaft 202 proximal to, aligned with, and distal to the guidewire port 220.

The port brace 506 can stiffen the region of the catheter shaft 202 around the guidewire port 220. The port brace 506 may add material to the port region, and thus, can increase a column strength of the catheter shaft 202 in that region. For example, the port brace 506 may provide additional material in the port region, even when the port brace 506 is formed from a material that is more flexible than the catheter shaft material, e.g., a polymer having a durometer less than Shore 75D. Alternatively or additionally, the particular materials used to form the port brace 506 may be stiffer than the material used to form the catheter shaft 202. For example, the catheter shaft 202 may be extruded from a polymer, such as a polyurethane having a durometer of Shore 75D. By contrast, the port brace 506 may be fabricated from a comparatively stiffer steel material, e.g., SAE 304 stainless steel and/or 17-7 spring steel. Accordingly, the port brace 506 may be stiffer than the catheter shaft 202 in the port region, and the port brace 506 can stiffen the overall shaft structure in the port region.

In an embodiment, the proximal brace section 508 is stiffer than the distal brace section 510. For example, as described in more detail below, the proximal brace section 508 may have a solid cross-sectional area, e.g., formed by a mandrel inserted into and filling a lumen of a tube, and the distal brace section 510 may have an annular cross-sectional area, e.g., formed by a slotted tube having an open central channel. The solid cross-sectional area can be stiffer than the annular cross-sectional area and, thus, a stiffness of the port brace 506 can reduce in the distal direction 514 (from the proximal brace section 508 to the distal brace section 510).

In addition to, or instead of, the brace stiffness varying over each section, one or more of the brace sections may similarly have a stiffness variation over its length. For example, a stiffness of the distal brace section 510 may decrease in the distal direction 514. As described below, the stiffness gradation may be attributed to various structural features, such as a slotted tube, tapered tube, etc. In any case, a stiffness of the port brace 506 at a distal brace end 534 of the brace may be lower than a stiffness of the port brace 506 at a location proximal to the distal brace end 534.

In addition to reinforcing the port region to prevent kinking of the catheter shaft 202 during device delivery, the port brace 506 can include features to direct the guidewire 304 through the ports into the surrounding environment 222. In an embodiment, the port brace 506 includes the guidewire ramp 404. The guidewire ramp 404 can include a surface of the port brace 506 oriented obliquely relative to a longitudinal axis 520 of the guidewire lumen 308. For example, the oblique surface of the guidewire ramp 404 can extend within a plane oriented at a minimum angle of about 10-20°, e.g., 15°, relative to the longitudinal axis 520. Accordingly, the guidewire 304 can be directed proximally through the guidewire lumen 308 along the longitudinal axis 520, and deflect over the oblique surface of the guidewire ramp 404 to exit through the guidewire port 220 into the surrounding environment 222.

The distal brace section 510 reinforces the catheter shaft 202 distal to the guidewire port 220 and reduces a likelihood of shaft buckling. Abrupt changes in stiffness may promote buckling and therefore the distal brace section 510 can provide a stiffness transition into the catheter shaft 202. In an embodiment, the tissue treatment catheter 102 includes an isolation tube 530 extending through the guidewire lumen 308. The isolation tube 530 can be a tubular component extending distally through the guidewire lumen 308 and the balloon 112 to the guidewire support tip 302. More particularly, the isolation tube 530 can include a distal tube end located at the guidewire support tip 302 and a proximal tube end 532 in the guidewire lumen 308 near the distal brace end 534. For example, the proximal tube end 532 of the isolation tube 530 can be located adjacent to, e.g., immediately distal to, the guidewire port 220. A distance between the distal brace end 534 and the proximal tube end 532 may be small enough to avoid creating a kink point. For example, the proximal tube end 532 may abut the distal brace end 534. Alternatively, the distance between the distal end of the port brace 506 and the proximal end of the isolation tube may be less than 5 mm, e.g., 1 mm or less.

A stiffness transition between the distal brace section 510 and the isolation tube 530 may also be facilitated by creating a longitudinal overlap between the port brace 506 and the isolation tube 530. For example, in an embodiment, the proximal tube end 532 may be located proximal to the distal brace end 534. Locating the proximal tube end 532 proximal to the distal brace end 534 can be achieved by inserting the isolation tube 530 into a brace lumen 533 of the distal brace section 510. The brace lumen 533 can be a central lumen through which a guidewire may be tracked when the port brace 506 is in the guidewire lumen 308 and coaxially aligned with the isolation tube 530. More particularly, the brace lumen 533 can be coaxial with the guidewire lumen 308. When the isolation tube 530 is inserted within the distal brace section 510, an overlap between the components is formed. The overlap may be less than a distance between the distal port edge 536 of the guidewire port 220 and the distal brace end 534. For example, the overlap may be less than about 0.08 inch, e.g., about 0.01 inch.

Whether the proximal tube end 532 is proximal to or distal to the distal brace end 534, the proximal tube end 532 may be located distal to the guidewire port 220. For example, the proximal tube end 532 may be at least about 0.050 inch, e.g., 0.1 inch, distal to the distal port edge 536. Spacing the isolation tube 530 apart from the guidewire port 220 can allow the distal brace section 510 to transition the stiffness of the catheter shaft 202 smoothly between the guidewire port 220 and the isolation tube 530.

The isolation tube 530, like the port brace 506, can impart stiffness to the catheter structure. In an embodiment, the isolation tube 530 can include an internal support wire 540, such as a helical coil or a wire braid. The internal support wire 540 can impart bending stiffness to resist kinking of the catheter shaft 202 over a length between the port brace 506 and balloon 112. More particularly, the internal support wire 540, and the isolation tube structure generally, can provide strain relief for the section of the catheter shaft 202 within which the isolation tube 530 is located.

Having described the port region of the catheter shaft 202, including the port brace 506, generally, particular embodiments of port braces 506 are now described. It will be appreciated that these embodiments are provided by way of example and not limitation. Features of the individual embodiments may be combined to form a port brace having the characteristics described above. More particularly, the port brace may be embodied as a structure having the proximal brace section 508 that is stiff but resilient, and the distal brace section 510 that is less stiff than the proximal brace section 508 and/or reduces in stiffness in the distal direction 514.

Referring to FIG. 6, a side view of a port brace is shown in accordance with an embodiment. The port brace 506 can have a two-part structure. In an embodiment, the two-part structure includes a brace mandrel 602 inserted into a brace tube 604. Geometrical features of the port brace components are described in more detail below, however, it will be appreciated at this point that the brace mandrel 602 may be an elongated structure, such as a rod, extending from a proximal mandrel end 605 to a distal mandrel end 606. The distal mandrel end 606 can include the guidewire ramp 404. More particularly, the guidewire ramp 404 may be located at the distal mandrel end 606. As shown in FIG. 5, the brace mandrel 602 can extend along the guidewire lumen 308 proximal to the guidewire port 220 to add stability and strength to the tissue treatment catheter shaft 202.

FIG. 6 includes a line break in the brace mandrel 602. The brace mandrel 602 may be substantially longer than the brace tube 604. More particularly, the brace mandrel 602 may be several times longer than the brace tube 604. For example, the brace tube 604 may have a length of about 0.30-0.50 inch, e.g., about 0.40 inch, to extend through the guidewire lumen 308 within the port region. By contrast, the brace mandrel 602 may have a length to extend through the guidewire lumen 308 from the port region to a proximal mandrel end 605 located near the proximal catheter end 204. For example, the brace mandrel 602 of may have a length of about 20-70 inches, e.g., about 30 inches, such that the proximal mandrel end 605 is located just distal to the connectors of the tissue treatment catheter 102. For example, the proximal mandrel end 605 may be immediately adjacent to a hub 250 (FIG. 2) connecting the fluid ports 210 and/or the external connector 212 to the catheter shaft 202. The brace mandrel 602 may therefore run up to the proximal hub 250 of the catheter shaft 202 to support and strengthen the catheter shaft 202 over a length between the guidewire port 220 and the hub 250.

The brace tube 604 may be formed from a hypotube. The hypotube can be laser cut or otherwise machined to introduce features into the tubular structure. For example, a semi-cylindrical portion of a wall of the tubular structure may be removed to form a guidewire notch 610. The guidewire notch 610 can extend over the medial brace section 512. The cross-sectional profile of the brace tube 604 along the guidewire notch 610 can reveal a semi-circular annulus. More particularly, the wall of the brace tube 604 can be C or U-shaped along the guidewire notch 610. The partial wall section along the guidewire notch 610 can define a reinforcement bridge 612 of the brace tube 604. The reinforcement bridge 612 can connect the proximal brace section 508 to the distal brace section 510.

In an embodiment, the brace tube 604 has a proximal collar 614 within the proximal brace section 508. The proximal collar 614 can be a tubular section of the brace tube 604, which is mounted on the brace mandrel 602. For example, the proximal collar 614 can be loaded onto the brace mandrel 602 such that the collar is proximal to the guidewire ramp 404. More particularly, the guidewire ramp 404 may be located distal to the proximal collar 614, e.g., aligned with the brace notch, such that the guidewire 304 will ride over the guidewire ramp 404 to exit the brace tube 604 through the guidewire notch 610. The proximal collar 614 may be welded or otherwise bonded to the brace mandrel 602 to secure the brace tube 604 to the brace mandrel 602.

The reinforcement bridge 612 extends distally from the proximal collar 614 to a distal tube segment 616. The distal tube segment 616 may be within the distal brace section 510, and can have a lower stiffness than the proximal brace section 508, e.g., the brace mandrel 602 and/or the combination of the proximal collar 614 welded on the brace tube 604. Similarly, the proximal brace section 508, which includes the brace mandrel 602 and the proximal collar 614, may be stiffer than the reinforcement bridge 612. In an embodiment, the reinforcement bridge 612 connecting the proximal collar 614 to the distal tube segment 616 defines the guidewire notch 610. When located within the guidewire lumen 308 (FIG. 5) the guidewire notch 610 can be aligned with the guidewire port 220 between the guidewire ramp 404 and the distal tube segment 616.

The brace tube 604 can have a slot 618 within the distal brace section 510. The slot 618 can weaken the distal tube segment 616, causing the distal tube segment 616 to have a lower stiffness than, e.g., the proximal collar 614 that has a full annular cross-section. Particular embodiments of the slot 618 are described in more detail below.

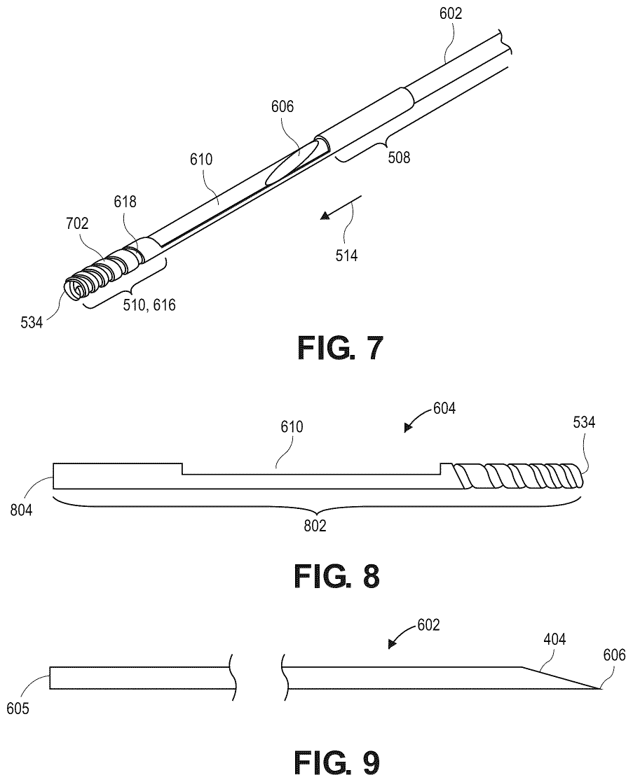

Referring to FIG. 7, a perspective view of a port brace is shown in accordance with an embodiment. It will be appreciated, based on the above description, that the port brace 506 has the rigid proximal brace section 508, the guidewire notch 610 aligned with the guidewire ramp 404 for the guidewire 304 to leave the guidewire lumen 308 out of the guidewire port 220, and the distal brace section 510 that is more flexible than the proximal brace section 508.

In an embodiment, the distal brace section 510 includes the distal tube segment 616 extending in the distal direction 514 from the guidewire notch 610 to the distal brace end 534. In addition to being less stiff than the proximal brace section 508, a stiffness of the distal brace section 510 can decrease in the distal direction 514. The stiffness of the distal tube segment 616, either relative to the proximal brace section 508 or over its own length, may be influenced by: the materials used to form the port brace 506, slot 618 structures within the brace tube 604, or cross-sectional area characteristics of the port brace 506.

The stiffness of the port brace 506 along its length may correspond to the materials used within each brace section. In an embodiment, the port brace 506 may be fabricated from materials that are inexpensive and easy to work with. For example, the port brace 506 may be formed from alloys of stainless steel. Alternatively, nickel titanium may be used to manufacture the port brace 506. Nickel titanium possesses an elastic modulus that reduces a likelihood of brace kinking, when compared to other medical grade materials, however, nickel titanium can be expensive. Accordingly, at least a portion of the port brace 506 may be formed from a stainless steel alloy having resilience that allows the port brace 506 to recover from bending.

In an embodiment, the brace mandrel 602 is fabricated from an alloy of stainless steel that is more resilient and/or has a higher elastic modulus than an alloy of stainless steel used to fabricate the brace tube 604. For example, the brace mandrel 602 may be fabricated from 17-7 spring steel and the brace tube 604 may be fabricated from SAE 304 stainless steel alloy. Accordingly, the brace mandrel 602 may be stiff enough to reduce the likelihood of catheter buckling, but resilient enough to bend under a load from a straightened shape and recover to the straightened shape when the load is removed.

The distal tube segment 616 can include the slot 618 to reduce the stiffness of the section relative to a solid annular wall. For example, the slot 618 used to form the brace slot can be a spiral cut slot. The slot 618 can extend spirally about the longitudinal axis 520 of the guidewire lumen 308 (or the central lumen of the brace tube 604) to define a helical wall 702. The slot 618 can have a pitch. For example, a spiral path of the slot 618 can have a pitch that varies over a length of the distal tube segment 616.

In an embodiment, the slot 618 has a constant pitch to define the helical wall 702 having a uniform stiffness over a length of the distal tube segment 616. When the pitch of the slot 618 is constant, a width, e.g., a longitudinal distance, of the helical wall 702 can be constant. More particularly, each turn of the helical wall 702 can have a same width measured in the distal direction 514. Although the stiffness of the distal tube segment 616 may be constant over its length, the distal tube segment 616 stiffness can be less than the proximal brace section 508.

The distal tube segment 616 may also include a slot 618 having a variable pitch. The variable pitch may be defined by a midline of the slot 618 that changes in the distal direction 514. For example, the pitch may decrease in the distal direction 514. The pitch of the slot 618 corresponds to the width of the helical wall 702. Each turn of the helical wall 702 is located between adjacent turns of the slot 618 and, accordingly, when the pitch of the slot 618 increases or decreases, the width of the helical wall 702 correspondingly increases or decreases. Accordingly, when the pitch of the slot 618 decreases, the width of the helical wall 702 decreases, and the stiffness of the distal tube segment 616 at the location of the helical wall 702 also decreases. More particularly, the pitch of the slot 618 can decrease in the distal direction 514 to reduce the stiffness of the brace tube 604 in the distal direction 514. The brace tube 604 can therefore transition to a lower stiffness in the distal direction 514 to avoid abrupt changes in stiffness between the distal tube segment 616 at the distal brace end 534 and the catheter shaft 202. The distal region of the port brace 506 can therefore provide a transitional stiffness to the tissue treatment catheter 102. Smoothly transitioning the stiffness over the shaft length can reduce the likelihood of kinks occurring during device delivery.

In an embodiment, the variable pitch of the slot 618 immediately adjacent to the guidewire notch 610 is in a range of about 0.025-0.035 inch, e.g., about 0.031 inch. At a distance of about 0.05 inch from the guidewire notch 610, the variable pitch of the slot 618 can decrease to be within a range of about 0.010-0.020 inch, e.g., about 0.016 inch. At a distance of about 0.10 inch from the guidewire notch 610, the pitch of the slot 618 can decrease to be within a range of about 0.005 to 0.010 inch, e.g., about 0.008 inch. Accordingly, a pitch of the slot 618 can decrease consistently and substantially to transition the tube stiffness from being relatively stiff near the guidewire notch 610 to being relatively flexible near the distal brace end 534.

The stiffness of the distal tube segment 616 may vary based on structural characteristics in addition to or instead of the slotted tube configuration. For example, a cross-sectional area of the distal brace section 510 may decrease in the distal direction 514. The decrease in the cross-sectional area can be related to the changing slot pitch. For example, as described above, when the pitch decreases, the helical wall 702 width decreases and, thus, a material volume per unit length in the distal direction 514 also decreases. Alternatively or additionally, the annular profile of the helical wall 702 may have a cross-sectional area that decreases in the distal direction 514. For example, a cross-sectional thickness of the annular profile of the brace tube 604 may decrease in the distal direction 514. The annular profile may be defined by an outer diameter and an inner diameter of the brace tube 604. An outer surface and/or an inner surface of the brace tube 604 can be machined, e.g., grinded, to introduce a taper in the cross-sectional area. More particularly, the cross-sectional area can decrease in the distal direction 514. The reduced annular thickness results in a reduced bending stiffness. Accordingly, the stiffness of the distal tube segment 616 may vary and reduce in the distal direction 514.

Referring to FIG. 8, a side view of a brace tube of a port brace is shown in accordance with an embodiment. The brace tube 604 can have a tube length 802 between the distal brace end 534 and a proximal tube end 804. The guidewire notch 610 can be formed in the brace tube 604 at a location between the distal brace end 534 and the proximal tube end 804. As described above, the guidewire notch 610 can align with guidewire port 220. The tube length 802 can be longer than the guidewire port 220. For example, the guidewire port 220 may have a length of about 0.20-0.30 inch, and the tube length 802 may be in a range of about 0.30-0.50 inch, e.g., about 0.40 inch. By contrast, the guidewire notch 610 may have a length similar to the guidewire port 220. For example, the guidewire notch 610 can have a length in a range of about 0.15-0.25 inch, e.g., about 0.20 inch. The brace tube 604 may be formed from any biocompatible and bioburden proof material, such as, 316 stainless steel, Nitinol, PEEK plastics, SAE 304 stainless steel alloy, etc.

Referring to FIG. 9, a side view of a brace mandrel of a port brace is shown in accordance with an embodiment. The brace mandrel 602 can be a solid rod extending from the proximal mandrel end 605 to the distal mandrel end 606 in the distal direction 514. The brace mandrel 602 can have a diameter facilitating a press or slip fit with an inner dimension of the brace tube 604. For example, the central lumen of the brace tube 604 and the outer diameter of the brace mandrel 602 can be substantially equal. In an embodiment, the matching diameter can be in a range of about 0.015-0.020 inch, e.g., about 0.016 inch. In any case, an outer diameter of both the brace mandrel 602 and brace tube 604 can be less than a diameter of the guidewire lumen 308 to allow the components to fit within the guidewire lumen 308. For example, the guidewire lumen 308 can have a diameter of about 0.021 inch and the outer diameter of the brace mandrel 602 and/or the brace tube 604 may be less than or equal to about 0.0195 inch. The brace mandrel 602, like the brace tube 604, may be formed from any biocompatible and bioburden proof material, such as, 316 stainless steel, Nitinol, PEEK plastics, SAE 304 stainless steel alloy, etc.

The brace mandrel 602 can be located within the guidewire lumen 308, and can extend over a length that accommodates a type of vascular access used to deliver the tissue treatment catheter 102 to a target anatomy. For example, the tissue treatment catheter 102 may be delivered to the target anatomy via a femoral access point or via a radial access point. The length of the tissue treatment catheter 102 required to reach the target anatomy can differ based on the access point used. The catheter may need to be longer to reach the target anatomy when delivered through the radial access point than when delivered through the femoral access point. When the catheter length is increased, the brace mandrel 602 may be similarly lengthened. For example, a brace mandrel 602 used in a tissue treatment catheter 102 configured for femoral access may be 30 inches long, and a brace mandrel 602 used in a tissue treatment catheter 102 configured for radial access may be 54 inches long. The brace mandrel 602 can therefore stiffen the catheter shaft 202 over a length appropriate for the intended use.

Referring to FIG. 10, a cross-sectional view, taken about line A-A of FIG. 5, is shown in accordance with an embodiment. The cross-section may be taken within the proximal brace section 508. Furthermore, when referring to the proximal brace section 508 having a two-part design, the cross-section may be taken proximal to the brace tube 604. Accordingly, in the two-part design the cross section may be taken at a location that only includes the brace mandrel 602 within the guidewire lumen 308. As described above, the proximal brace section 508 can have a solid cross-sectional profile 1002. For example, the brace mandrel 602 can include a rod having a cross-sectional area. The brace mandrel 602 can be located within the guidewire lumen 308, and may be smaller than the lumen size.

The catheter shaft 202 can have a multi-lumen configuration, which includes the guidewire lumen 308 and the fluid lumen 502 previously referred to. In an embodiment, the catheter shaft 202 includes three or more lumens. For example, in addition to the guidewire lumen 308 and the fluid lumen 502, the catheter shaft 202 can include one or more of a second fluid lumen 1004 or a cable lumen 1006.

The second fluid lumen 1004 may be used to communicate fluid to or from the interior 310 of the balloon 112. More particularly, the inflation fluid 111 can be circulated through the interior 310 by advancing fluid in the distal direction 514 through one of the fluid lumen 502 or the second fluid lumen 1004, and aspirating inflation fluid 111 in a proximal direction through the other of the fluid lumen 502 or the second fluid lumen 1004.

The cable lumen 1006 can provide a space to contain electrical cables. The electrical cables can advance through the catheter shaft 202 within the cable lumen 1006 to communicate electrical signals from the external connector 212 to the transducer 208. More particularly, the electrical cable(s) in the cable lumen 1006 can be electrically connected to the ultrasound transducer 208. One or more electrical cables can be housed within the cable lumen 1006. For example, a signal cable and a ground cable may be placed adjacently within the cable lumen 1006.

Referring to FIG. 11, a cross-sectional view, taken about line B-B of FIG. 5, is shown in accordance with an embodiment. The cross-section may be taken through the proximal brace section 508. More particularly, the cross-section may be taken at a location of the proximal brace section 508 having both the brace mandrel 602 and the brace tube 604. The proximal brace section 508 can have a solid cross-sectional profile 1002 in that an annular cross-sectional profile 1102 of the brace tube 604 can combine with the solid cross-sectional profile 1002 of the brace mandrel 602 to form a solid composite cross-sectional area. In an embodiment, the brace tube 604 and the brace mandrel 602 interface at a joint 1104. The joint 1104 can be a material and/or surface interface between the components. For example, the joint 1104 can include an adhesive or thermal weld between the brace mandrel 602 and the brace tube 604. Alternatively, the brace mandrel 602 and the brace tube 604 may be joined by an interference fit, e.g., a press fit, along apposing surfaces at the joint 1104. The solid cross-sectional profile 1002 of the brace mandrel 602 and the annular cross-sectional profile 1102 of the brace tube 604 can be concentrically disposed about the longitudinal axis 520 of the guidewire lumen 308. It will be appreciated that the distal brace section 510 can include a section of the brace tube 604 having a same annular cross-sectional profile 1102 as a section of the brace tube 604 within the proximal brace section 508 and, thus, the distal brace section 510 can have the annular cross-sectional profile 1102.

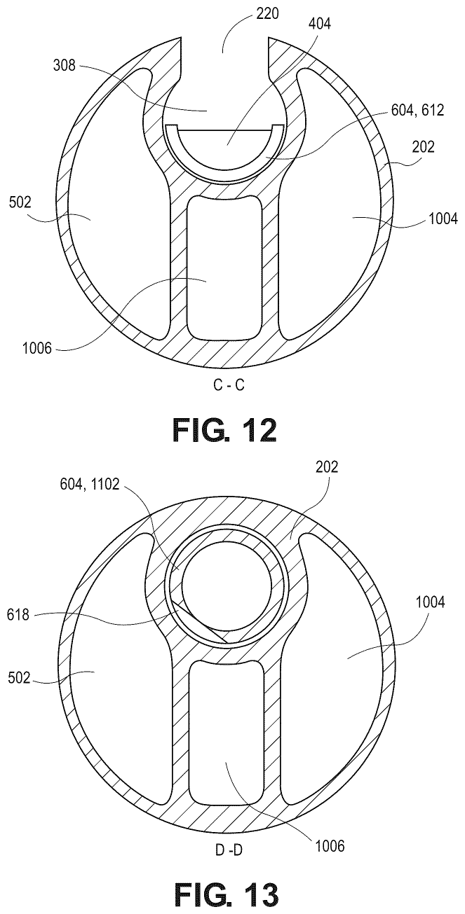

Referring to FIG. 12, a cross-sectional view, taken about line C-C of FIG. 5, is shown in accordance with an embodiment. The cross section can be taken within the medial brace section 512 of the port brace 506. More particularly, the cross-section may be taken through the guidewire ramp 404 of the port brace 506. In cross-section, guidewire ramp 404 is represented as having a flat top surface and a semicircular area. Similarly, the brace tube 604 surrounding the guidewire ramp 404 can have a semicylindrical, or U-shaped, cross-sectional area. As shown, the port brace 506 can appose the guidewire port 220 to direct the guidewire 304 from the guidewire lumen 308 toward the surrounding environment 222.

The cross-sectional wall of the brace tube 604 is shown as being solid below the guidewire ramp 404. It will be appreciated, however, that the brace tube 604 can include one or more slits or openings along the medial brace section 512 to enhance the flexibility of the reinforcement bridge 612. For example, the reinforcement bridge 612 can have holes, slits, zig-zag cuts, etc., to reduce the material over the medial brace section 512. The reinforcement bridge 612 may therefore further transition the stiffness from the stiffer proximal brace section 508 to the more flexible distal brace section 510.

Referring to FIG. 13, a transverse cross-sectional view, taken about line D-D of FIG. 5, is shown in accordance with an embodiment. FIG. 13 illustrates an annular cross-sectional profile of the distal brace section 510. The cross-section may be taken at a location of the distal brace section 510 having slots 618 in the brace tube 604. The annular cross-sectional profile may be partially interrupted by the slot 618. For example, the profile may include a C-shaped wall profile having a section removed. In FIG. 13, the section is between a six o'clock and a nine o'clock position, however, the section would be located at different angular clocking depending on longitudinal location of the cross-section. More particularly, the helical slot 618 would be located at different radial locations depending on the longitudinal location of the cross-section. In any case, the profile may be fully annular (circular) or primarily annular (C-shaped) and may extend along a greater portion of a circular wall than the semicylindrical, or U-shaped, cross-sectional area of the guidewire ramp 404.

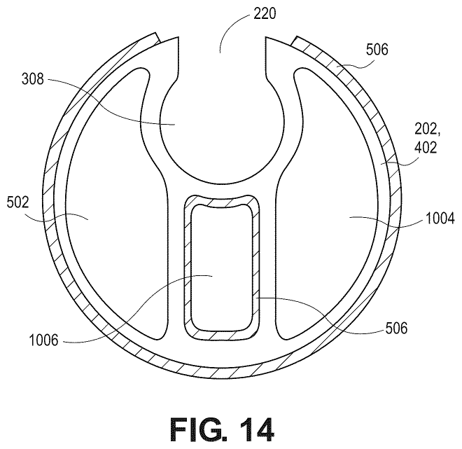

Referring to FIG. 14, a cross-sectional view of a tissue treatment catheter having a port brace in a non-guidewire lumen is shown in accordance with an embodiment. The tissue treatment catheter 102 can include a port brace 506 located outside of the guidewire lumen 308. Several port braces 506 are illustrated as being used instead of the port brace 506 within the guidewire lumen 308, however, such port braces 506 may be used alone or in addition to the port brace 506 located within the guidewire lumen 308.

In an embodiment, the port brace 506 is located in one or more of the fluid lumen 502, the second fluid lumen 1004, or the cable lumen 1006. For example, the port brace 506 can include a stiffening sleeve within the cable lumen 1006 aligned with the guidewire port 220. The stiffening sleeve may be a mesh or braided tubular structure extending longitudinally through the cable lumen 1006. The tubular structure can surround the cables stored within the cable lumen 1006. The stiffening sleeve mesh may be formed from metallic or polymeric strands. In an embodiment, the port brace 506 is stiffer than the outer shaft wall 402 over the length of the guidewire port 220. Accordingly, the port brace 506 can support and stiffen the catheter shaft 202 in the port region. The catheter shaft 202 may therefore be less likely to buckle under axial loads applied during device delivery.

The port brace 506 located outside of the guidewire lumen 308 may alternatively include a port brace 506 mounted on the outer shaft wall 402. For example, the port brace 506 can include an external sleeve that fits over the outer shaft wall 402. The sleeve can include an opening at a location aligned with the guidewire port 220. Accordingly, the stiffening sleeve can support the catheter shaft 202 over the port region and allow the guidewire 304 to pass through the guidewire port 220 toward the surrounding environment 222. The protective sleeve may have a mesh or braid structure as described above. Alternatively, the protective sleeve can include a thin-walled tubular structure mounted on the catheter shaft 202. For example, the externally-mounted port brace 506 can include a stiffening tube having a wall thickness in a range of about 0.0015-0.0025 inch, fabricated from a thermoplastic such as fluorinated ethylene propylene, polytetrafluoroethylene, polyethylene terephthalate, etc. In any case, the port brace 506 can be stiffer than the outer shaft wall 402 at the guidewire port 220. Accordingly, the port brace 506 can support and stiffen the catheter shaft 202 in the port region.

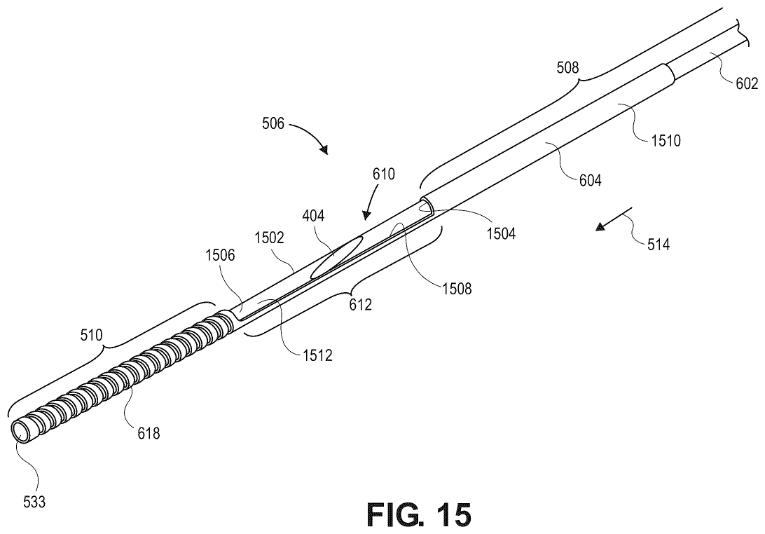

Referring to FIG. 15, a perspective view of a port brace is shown in accordance with an embodiment. The port brace 506 can include components similar to those described above. For example, the port brace 506 can include the proximal brace section 508 and the distal brace section 510. The reinforcement bridge 612 can extend longitudinally between the proximal brace section 508 and the distal brace section 510. As described above, the brace mandrel 602 can be used to add stability and strength to the port brace 506. More particularly, the brace mandrel 602 can be loaded into the brace lumen 533 within the proximal brace section 508, and the brace tube 604 can be fixed to the brace mandrel 602, e.g., by a thermal weld or adhesive joint. The brace mandrel 602 stiffens a portion of the brace tube 604.

A stiffness of the port brace 506 can transition in the distal direction 514. For example, the port brace 506 can include a rigid proximal section where the proximal brace section 508 overlaps the brace mandrel 602, a less rigid medial section having the guidewire notch 610, defined by a notch edge 1502, to allow a guidewire 304 to exit from the brace lumen 533 through the guidewire notch 610, and a spring-like tip, e.g., the distal brace section 510 having the slot 618 in the wall for increased flexibility.

The guidewire notch 610 can be defined by a notch edge 1502. More particularly, the notch edge 1502 can surround the guidewire notch 610, defining sides of the edge, as well as a proximal notch edge 1504 and a distal notch edge 1506. As shown in FIG. 5, the guidewire ramp 404 of the brace mandrel 602 can be located distal to the proximal notch edge 1504. In an embodiment, a distance between the guidewire ramp 404 and the proximal notch edge 1504 can be increased to strengthen a proximal portion of the reinforcement bridge 612. More particularly, the guidewire ramp 404 may be in a more distal position in FIG. 15, relative to the proximal notch edge 1504, than is shown in FIG. 5.

The distance between the guidewire ramp 404 and the proximal notch edge 1504 may be predetermined. By way of example, the distance can be in a range of 0.60 to 3.18 inches. Nominally, the distance may be 1.905 inches. By extending the brace mandrel 602 within the brace tube 604, an amount of the cut section is reduced. More particularly, a distance between the guidewire ramp 404 and the distal notch edge 1506 can be correspondingly reduced.

Reducing the length of the unsupported reinforcement bridge 612 in such a manner can reduce kinkability and improve buckling resistance of the assembly.

Integrity of the port brace assembly can also be improved by securing the brace tube 604 relative to the brace mandrel 602. The brace tube 604, which is mounted on the brace mandrel 602, can be secured to the brace mandrel 602 by a joint. The joint may, for example, be a thermal joint or an adhesive joint. In an embodiment, the port brace 506 includes a weld 1508 between the brace tube 604 and the brace mandrel 602. For example, the weld 1508 can be along the notch edge 1502 that defines the guidewire notch 610. A seam or interface can exist between the brace mandrel 602 and the brace tube 604 along the notch edge 1502. The weld 1508 can be located in and/or along the seam to unify the brace components and prevent relative movement at the weld location. For example, the weld 1508 may be located along the proximal notch edge 1504 to resist movement of the brace mandrel 602 and the brace tube 604 at that location. Similarly, the weld 1508 may extend distally from the proximal notch edge 1504, along a side portion of the notch edge 1502, to affix the components. The weld 1508 may extend along the seam up to or before the guidewire ramp 404. For example, the weld 1508 can extend to a distal weld end at a location that is 0.005 inch proximal to the guidewire ramp 404. The welded brace tube 604 can resist sticking out and or bending away from the brace mandrel 602 when a user deforms the assembly, or when the tissue treatment catheter 102 tracks through an anatomical curve. More particularly, the bond along the seam can reduce a likelihood that the brace tube 604 will separate from the brace mandrel 602 when a bending load is applied to the assembly.

The port brace 506 can be adhered to the catheter shaft 202. For example, the catheter shaft 202 may be formed at least in part from a polymer that is thermally bonded to the port brace 506. Thermal bonding may be through a reflowing operation, in which the catheter shaft 202 is heated and reflowed over an outer surface 1510 of the port brace 506. The port brace 506 can have surface characteristics to promote adhesion to the catheter shaft 202. In an embodiment, the outer surface 1510 of the port brace 506 is roughened. For example, the outer surface 1510 can be knurled, laser abraded, or subjected to another roughening process. As a result of the roughening, the outer surface 1510 can be rougher than an inner surface 1512 of the brace tube 604. The inner surface 1512 can define the brace lumen 533. The inner surface 1512 can have a surface roughness that is less than a roughness of the outer surface 1510, which outer surface roughness may be in a range of 50-150 micron, e.g., 100 micron. The surface roughness is defined by microscopic peaks and valleys in the outer surface 1510 that increase friction between the port brace 506 and the catheter shaft 202. Accordingly, bonding between the port brace 506 and the catheter shaft 202 can be enhanced.

Bonding between the catheter shaft 202 and the port brace 506 may be enhanced by increasing surface area contact in other ways. For example, a length of the port brace 506 can be increased to increase surface contact between the components and thereby improve adhesion.

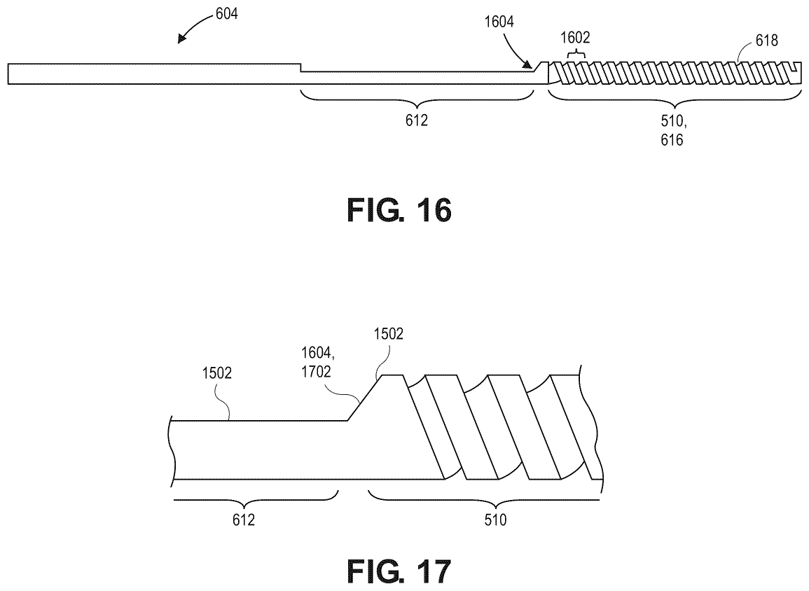

Referring to FIG. 16, a side view of a brace tube of a port brace is shown in accordance with an embodiment. As described above, the distal brace section 510 can include the slot 618. The slot 618 can be a spiral cut slot, e.g., a helical slot, extending along a length of the brace section. The slot 618 can include a pitch, which may be variable, as described above. Alternatively, the pitch may be constant. For example, the distance between adjacent turns in the spring-like structure of the distal tube segment 616 can be the same. In an embodiment, the pitch is a fraction of a length of the distal tube section. For example, the distal tube section can have the length distal to the reinforcement bridge 612, and the pitch may be a fraction of the length. The length may be 0.24 inch, and the fraction may be 1/64. Accordingly, the pitch may be 0.004 inch, in an embodiment. Maintaining a constant pitch can improve manufacturability of the port brace 506 while providing sufficient stiffness transition to facilitate device tracking.

Referring to FIG. 17, a side view of a brace tube transition is shown in accordance with an embodiment. The stiffness of the brace tube 604 can transition at discrete locations. For example, the reinforcement bridge 612 may be stiffer than the spiral-cut distal brace section 510, and a transition point 1604 can be located between the sections. The transition point 1604 can include a bevel 1702, e.g., an angled surface connecting the reinforcement bridge 612 to the distal brace section 510. The bevel 1702 can be a portion of the notch edge 1502 along which the reinforcement bridge 612 transitions into the distal brace section 510. In an embodiment, the bevel 1702 is an angled surface set at an angle of 45 degrees to the side edge portion of the notch edge 1502. The bevel 1702 may have a length of, for example, 0.005 to 0.010 inch, e.g., 0.007 inch. The angled surface of the bevel 1702 can strengthen the transition point 1604, relative to the strength of an orthogonal transition between the sections. More particularly, the angle can distribute material stress such that failure at the transition point 1604 is less likely to occur.

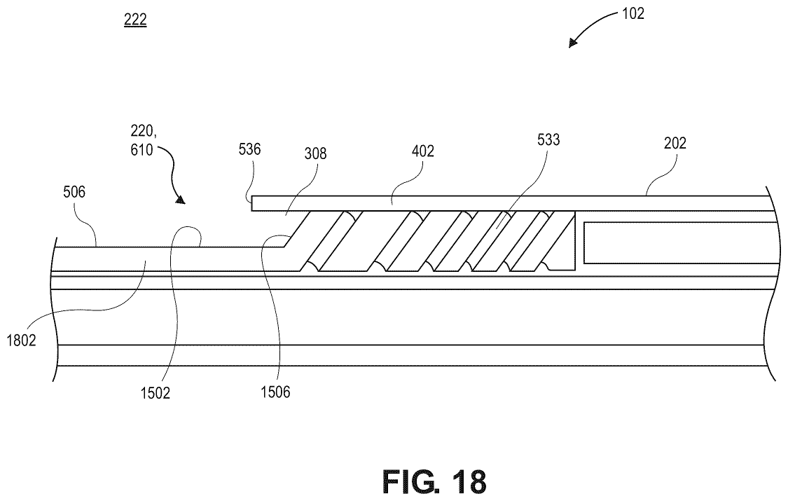

Referring to FIG. 18, a partial cross-sectional view of a tissue treatment catheter is shown in accordance with an embodiment. As described above, the tissue treatment catheter 102 can include the guidewire notch 610 and the guidewire port 220 in an aligned state. More particularly, the guidewire notch 610 can be longitudinally aligned with the guidewire port 220 such that a guidewire 304 can pass from the guidewire lumen 308 through the guidewire notch 610 and guidewire port 220 into the surrounding environment 222. Likewise, the guidewire notch 610 can be in the reinforcement bridge 612 and, thus, the reinforcement bridge 612 may be longitudinally aligned with the guidewire port 220.

The reinforcement bridge 612 can include a brace wall 1802 defining a solid portion of the reinforcement bridge 612. The brace wall 1802 can be opposite of the guidewire notch 610 and, thus, can be opposite of the guidewire port 220. Here, the term solid can refer to a lack of a discontinuity within the reinforcement bridge 612 anywhere other than the guidewire notch 610. More particularly, the guidewire notch 610 can be defined by the notch edge 1502 extending around the notch perimeter, and at no point on an opposite side of the edge from the notch is there a hole, slot, or other discontinuity along the brace wall 1802. The brace wall 1802 may, therefore, be a solid, curved wall having a semi-circular cross-sectional profile.

The port brace 506 may be assembled with the catheter shaft 202 to reduce a likelihood of friction points on the guidewire that passes through the brace lumen 533 and the guidewire lumen 308 into the guidewire notch 610 and the guidewire port 220. In an embodiment, the distal notch edge 1506 of the guidewire notch 610 is distal to the distal port edge 536 of the guidewire port 220. The longitudinal offset between the edges may be achieved, for example, by lengthening the guidewire notch 610 to position the spiral-cut distal brace section 510 more distal within the guidewire lumen 308. The longitudinal gap between the edges can reduce a likelihood that the guidewire 304 will rub on an inner surface of the port brace 506 when it extends proximally toward the guidewire port 220. More particularly, the guidewire 304 is more likely to rub against the polymeric catheter shaft 202 at the distal port edge 536 rather than the metallic port brace 506 at the distal notch edge 1506. Accordingly, any coating on the guidewire 304 or the port brace 506 is less likely to be scraped away during device tracking.

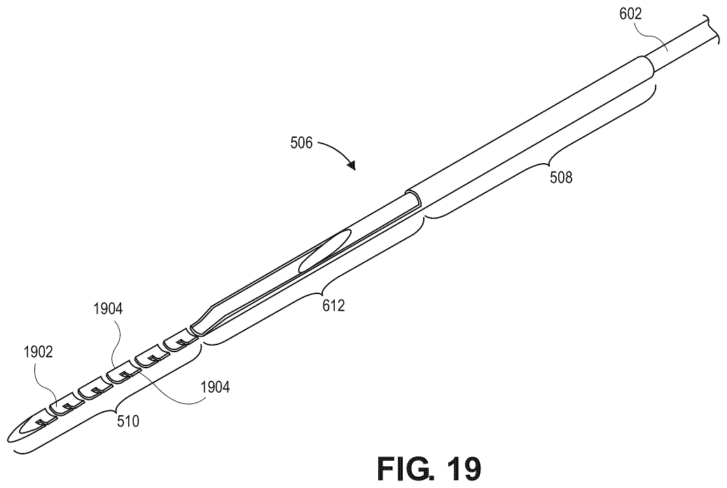

Referring to FIG. 19, a perspective view of a port brace is shown in accordance with an embodiment. The port brace 506 may include a distal brace section 510 that does not have a spiral or tubular structure. More particularly, the distal brace section 510 can include a spine structure to support the catheter shaft 202 within the guidewire lumen 308, and the spine structure may extend along one side of the lumen without extending circumferentially around the guidewire lumen 308.

In an embodiment, the port brace 506 includes the reinforcement bridge 612 extending between the proximal brace section 508 and the distal brace section 510. The reinforcement bridge 612, the proximal brace section 508, and the brace mandrel 602 can have structures similar to those described above. The distal brace section 510 can differ from the structure described above, however. For example, the distal brace section 510 can include a flexible partial-tube structure that defines an open channel rather than a closed lumen. The partial-tube-like structure can include a spine wall 1902 having lateral edges 1904.

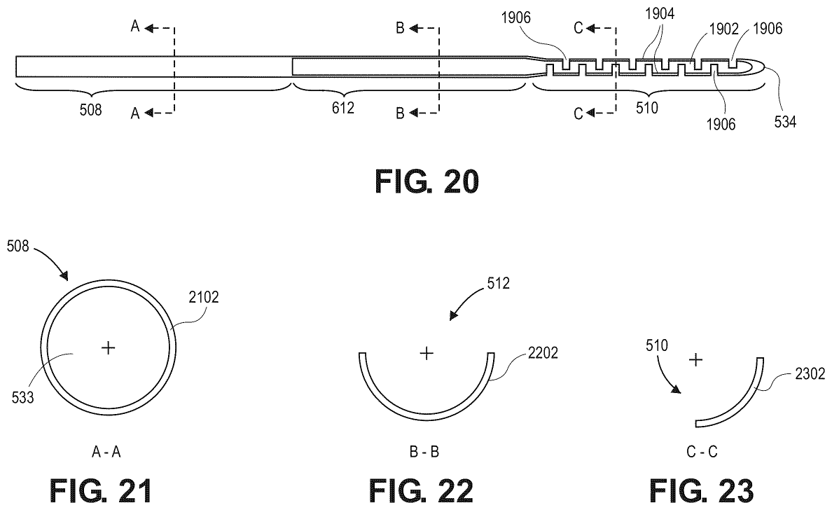

Referring to FIG. 20, a top view of a brace tube of a port brace is shown in accordance with an embodiment. The lateral edges 1904 of the distal brace section 510 can extend longitudinally and be offset from each other by a central channel. More particularly, the central channel can be above the spine wall 1902, between the lateral edges 1904. In an embodiment, the isolation tube 530 can extend through the central channel when the port brace 506 is assembled with the catheter shaft 202. The isolation tube 530 can be cradled by the distal brace section 510. The guidewire 304 can therefore extend proximally through the isolation tube 530 and exit upward through the guidewire notch 610 and the guidewire port 220 without scraping on a proximal edge of the distal brace section 510.

It will be appreciated that the partial-tube structure of the distal brace section 510 is similar to the structure of the reinforcement bridge 612. The distal brace section 510 may, however, be less stiff than the reinforcement bridge 612. In an embodiment, the distal brace section 510 includes a plurality of longitudinally offset cuts 1906 extending laterally inward through the spine wall 1902. Each of the radial cuts 1906 can locally reduce the stiffness of the spine wall 1902 without deforming the distal brace section 510. More particularly, the cuts 1906 can create a zig-zag brace pattern in which the spine wall 1902 undulates or zig-zags from the reinforcement bridge 612 to the distal brace end 534. The zig-zag structure has less material per unit length than the reinforcement bridge 612 and, thus, can be more flexible than the reinforcement bridge 612. The spine wall 1902 may, nonetheless, have sufficient rigidity to support the isolation tube 530 and resist buckling of the catheter shaft 202 when loaded axially or in bending.

Referring to FIG. 21, a cross-sectional view of a brace tube, taken about section line A-A of FIG. 20, is shown in accordance with an embodiment. The proximal brace section 508 can have a tubular wall 2102. The tubular wall 2102 can have a circular cross-sectional profile surrounding the brace lumen 533. The profile can therefore effectively have a 360 degree arc angle measured about a central axis. The tubular wall 2102 can have a larger cross-sectional area than the reinforcement bridge 612 and the distal brace section 510 and, thus, may be stiffer than those sections.

Referring to FIG. 22, a cross-sectional view of a brace tube, taken about section line B-B of FIG. 20, is shown in accordance with an embodiment. The reinforcement bridge 612 can have a first arc-shaped wall 2202. The first arc-shaped wall 2202 can have a u-shaped cross-sectional profile. The profile may extend about the central axis by less than a full circle. The profile can therefore define the open channel within an interior of the u-shape. For example, the profile may have an arc angle of 180 degrees. Accordingly, the reinforcement bridge 612 may be less stiff than the proximal brace section 508.

Referring to FIG. 23, a cross-sectional view of a brace tube, taken about section line C-C of FIG. 20, is shown in accordance with an embodiment. The distal brace section 510 can have a second arc-shaped wall 2302. The arc-shaped wall can replace the tubular structure (see, e.g., FIG. 7) with a backbone structure that supports the isolation tube 530 along one side.

The second arc-shaped wall 2302 can have a u-shaped cross-sectional profile. The profile may extend about the central axis by less than half a circle. The profile can therefore define the open channel within an interior of the u-shape. For example, the profile may have an arc angle of 90 degrees. In an embodiment, the first arc-shaped wall 2202 has a larger arc angle than the second arc-shaped wall 2302. Accordingly, the distal brace section 510 may be less stiff than the reinforcement bridge 612.

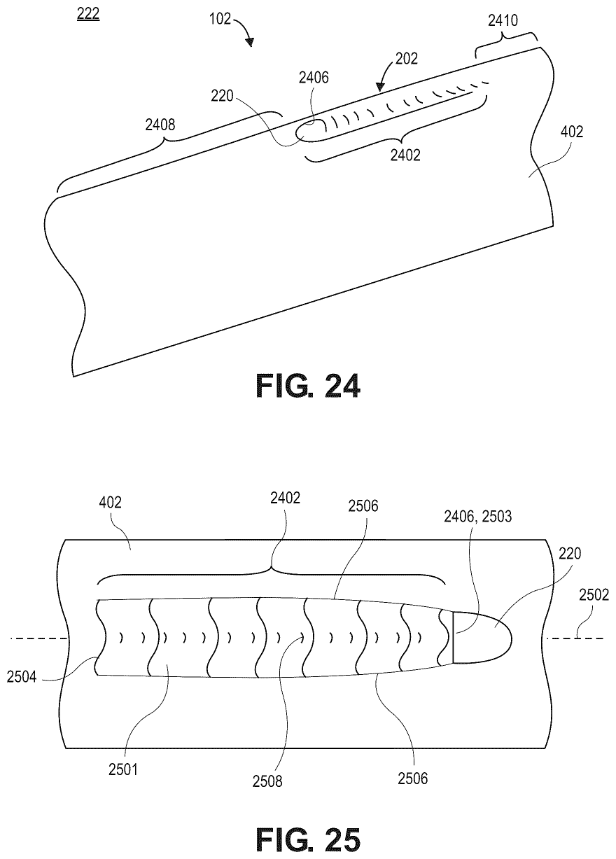

Referring to FIG. 24, a perspective view of a guidewire port of a tissue treatment catheter is shown in accordance with an embodiment. The catheter-based intraluminal tissue treatment catheter 102 can include a guidewire port 220 that does not incorporate the port brace, but nonetheless provides a supportive passage for the guidewire in a manufacturable platform. More particularly, as described below, the tissue treatment catheter 102 can include the catheter shaft 202 having a guidewire port 220 that may be easier and less expensive to manufacture than one or more of the embodiments described above, while achieving similar benefits.

In an embodiment, the catheter shaft 202 has a guidewire port 220 that includes a ramp formed from the native shaft material. The catheter shaft 202 can have the outer shaft wall 402 extending around the internal lumens of the tissue treatment catheter 102. More particularly, an outer surface of the outer shaft wall 402 can face radially outward and can extend, e.g., circumferentially, around the internal lumens such as the guidewire lumen 308, the fluid lumen(s) 502, or the cable lumen 1006. The outer shaft wall 402 can be processed, e.g., reflowed, to form the ramp. For example, the outer shaft wall 402 can have a collapsed section 2402 that is indented inward relative to the surrounding outer surface. Accordingly, the collapsed section 2402 can provide a tapered ramp from the guidewire lumen 308 to a surrounding environment 222.

A port edge 2406 can define the guidewire port 220 that extends through the outer shaft wall 402 into the guidewire lumen 308 into the guidewire lumen 308. The port edge 2406 may be formed by cutting, punching, skiving, or otherwise removing material from a tubular extrusion having the outer shaft wall 402. Accordingly, the port edge 2406 can be a continuous edge extending around a hole. As described below, the port edge 2406 may be deformed, e.g., may be pressed inward to form the collapsed section 2402 and, thus, the port edge 2406 may initially have an elliptical profile and may be deformed into a non-elliptical profile.

The collapsed section 2402 of the catheter shaft 202, which contains the ramp to direct the guidewire through the guidewire port 220, may be distinguished from adjacent sections of the catheter shaft 202 by a profile of the outer surface covering the sections. In an embodiment, the collapsed section 2402 is non-cylindrical. More particularly, a cross-sectional profile of the outer surface of the collapsed section 2402 can be non-circular. Such non-circularity can result from an indentation in the outer surface, as described below. By contrast, the outer shaft wall 402 can have a distal wall portion 2408 and a proximal wall portion 2410 on either side of the collapsed section 2402, one or both of which may be circular. More particularly, a cross-sectional profile of the outer surface of one or both of the distal wall portion 2408 distal to the collapse section or the proximal wall portion 2410 proximal to the collapsed section 2402 the collapsed section 2402 can be circular. Such circularity of the distal wall portion 2408, which may be distal to the port edge 2406, results from the distal wall portion 2408 being cylindrical. Similarly, the proximal wall portion 2410 can be proximal to the port edge 2406 and may be cylindrical. The cylindrical shaft wall sections can have continuous circular outer profiles on both sides (distal and proximal to) the non-circular outer profile of the ramp used to guide the guidewire through the guidewire port 220. The cylindricity of the catheter shaft 202 can promote trackability and low profile, and the non-cylindricity of the collapsed section 2402 can provide a guidewire ramp through the guidewire port 220.

Referring to FIG. 25, a top view of a guidewire port of a tissue treatment catheter is shown in accordance with an embodiment. A ramp 2501 formed by the collapsed section 2402 can extend longitudinally along the outer shaft wall 402. More particularly, the tissue treatment catheter 102 can include a longitudinal axis 2502, e.g., extending through a center of the guidewire lumen 308, a center of the catheter shaft 202, or a center of the cable lumen 1006. The longitudinal axis 2502 establishes a reference against which the surfaces of the catheter shaft 202 can be described. For example, when the longitudinal axis 2502 is a center of the catheter shaft 202, the cylindrical distal wall portion 2408 and proximal wall portion 2410 extend circumferentially about the longitudinal axis 2502.

In an embodiment, the collapsed section 2402 tapers outward from the port edge 2406 that surrounds the guidewire port 220. For example, a distal ramp end 2503 of the collapsed section 2402 at the port edge 2406 can be nearer to the longitudinal axis 2502 (into the page) than the collapsed section 2402 at a proximal ramp end 2504. More particularly, the proximal ramp end 2504 can be radially farther from the longitudinal axis 2502 than the distal ramp end 2503. The ramp 2501 can therefore taper outward in a proximal direction along the longitudinal axis 2502.

The ramp 2501 can have reference geometries, e.g., defining boundaries and/or surface features. For example, the ramp 2501 may be bounded by lateral boundaries 2506 separated from each other in a circumferential direction. More particularly, the lateral boundaries 2506 can define a lateral extent of the collapsed section 2402. Portions of the catheter shaft wall circumferentially outside of the lateral boundaries 2506 (on an opposite side of the boundaries from the ramp 2501) may be convex in a first radial direction relative to the longitudinal axis 2502. For example, the outside portions may be arc-shaped, e.g., cylindrical sections, convex outward away from the longitudinal axis 2502. By contrast, portions of the catheter shaft 202 between the lateral boundaries 2506 (on the ramp 2501) can be convex in a second radial direction relative to the longitudinal axis 2502. For example, the collapsed section 2402 can be convex inward (or concave outward) relative to the longitudinal axis 2502 of the catheter shaft 202. A bottom of the ramp 2501, e.g., a reference line defined by points along the ramp 2501 that are nearer to the longitudinal axis 2502 than other points on the ramp 2501 transversely aligned with the points, can provide a track along which the guidewire can slide when passing from the guidewire lumen 308 to the surrounding environment 222 through the guidewire port 220.

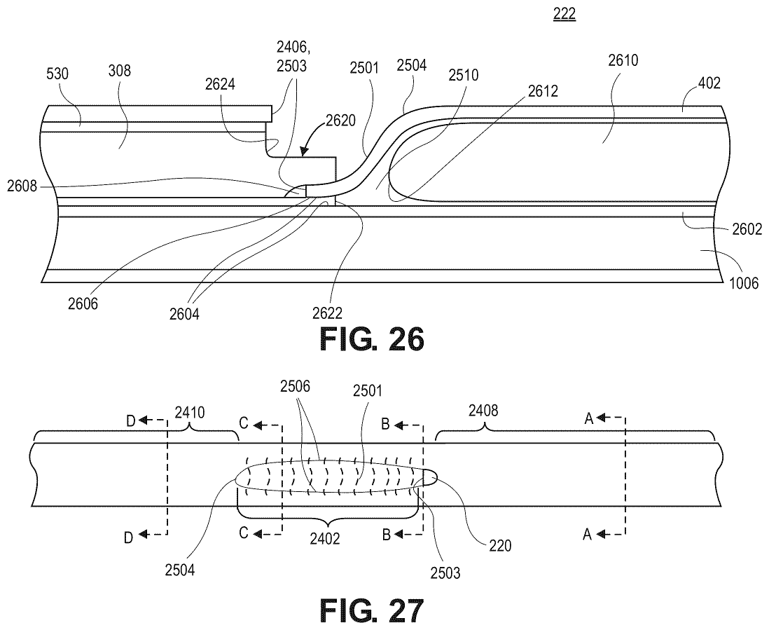

Referring to FIG. 26, a cross-sectional view of a guidewire port of a tissue treatment catheter is shown in accordance with an embodiment. Based on the above description, it is appreciated that the outers shaft wall has a collapsed section 2402 tapering outward from the port edge 2406. More particularly, the collapsed section 2402 provides the ramp 2501 that tapers upward from the distal ramp end 2503 at the port edge 2406 to the proximal ramp end 2504. Accordingly, the collapsed section 2402 is nearer to the cable lumen 1006 at the distal ramp end 2503 than at the proximal ramp end 2504.

In an embodiment, the catheter shaft 202 has the guidewire lumen 308 coaxially aligned with a stylet lumen 2510. The guidewire lumen 308 and the stylet lumen 2510 can be coaxially aligned. For example, the stylet lumen 2510 and the guidewire lumen 308 can be a same lumen prior to forming the ramp 2501. After the ramp 2501 is formed, however, the collapsed section 2402 (which defines the ramp 2501) can longitudinally separate the guidewire lumen 308 from the stylet lumen 2510. More particularly, the collapsed section 2402 can taper outward from the port edge 2406 longitudinally between the stylet lumen 2510 and the guidewire lumen 308, effectively forming a barrier between the lumens. The barrier acts as the ramp 2501 from the guidewire lumen 308 to the surrounding environment 222 while also reducing a likelihood of material or fluid passage between the guidewire lumen 308 and the stylet lumen 2510. Accordingly, when the guidewire passes through the guidewire lumen 308, it can pass along the ramp 2501 to exit through the guidewire port 220 into the surrounding environment 222, rather than continuing longitudinally into the stylet lumen 2510.

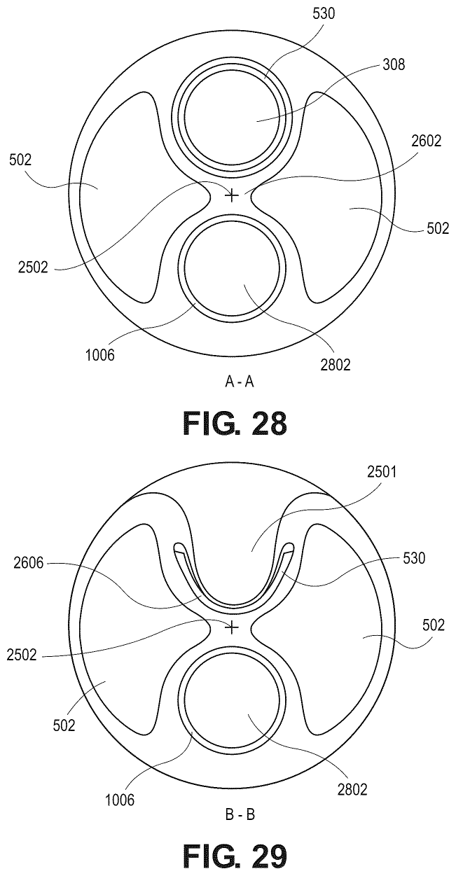

The barrier formed by the ramp 2501 between the guidewire lumen 308 and the stylet lumen 2510 can be formed by collapsing the portion of the outer shaft wall 402 surrounding the stylet lumen 2510 onto itself. For example, the port edge 2406, which can be formed when a hole is made in the cylindrical wall of the catheter shaft 202, can be forced inward against a septum 2602 separating the stylet lumen 2510 from the cable lumen 1006. More particularly, the collapsed section 2402 can include an inner wall 2604 having portions that appose each other at the port edge 2406. The inner wall 2604 at the port edge 2406 can be brought adjacent to itself in a u-shaped or arc-shaped double-wall (FIG. 28). Accordingly, the inner wall 2604 portions can appose each other along a seam 2606.

In an embodiment, the barrier between the stylet lumen 2510 and the guidewire lumen 308 can be perfected by a sealant 2608. More particularly, the sealant 2608 can be disposed along the seam 2606 to form a seal between the inner wall 2604 portions that can hermetically seal off the stylet lumen 2510 from the guidewire lumen 308. The seal may therefore prevent egress of blood or air between the guidewire lumen 308 and the stylet lumen 2510. The stylet 2610 can have a structure the same or similar as the brace mandrel 602. For example, the sealant 2608 may be an adhesive, such as a light-cured adhesive, which is dispensed along the seal and cured during a manufacturing process, as described below. the sealant 2608 may also be applied at a proximal end of the stylet lumen 2510 (not shown) to further seal off the stylet lumen 2510 and reduce a likelihood of fluid egress through the stylet lumen 2510 to or from the guidewire lumen 308.

The catheter shaft 202 can be supported by a stylet 2610 disposed in the stylet lumen 2510. The stylet 2610 may be an elongated member, such as a wire, inserted into the catheter to contribute rigidity to the catheter shaft 202. Accordingly, the stylet 2610 may be fabricated from a material that is stiffer than the outer shaft wall 402. For example, the catheter shaft 202 may be formed from a polymer and the stylet 2610 may be formed from a stainless steel. Alternatively, the components can both be polymers, with the stylet 2610 being a more rigid polymer.

The stylet 2610 can be disposed in the stylet lumen 2510 such that a distal stylet end 2612 abuts the ramp 2501. The distal stylet end 2612 may, for example, be a rounded tip of the stylet wire. The tip can be placed against the inner wall 2604 of the outer shaft wall 402 behind the ramp 2501. Accordingly, the stylet 2610 can act as a support to the ramp 2501. More particularly, when the guidewire is tracked through the guidewire lumen 308 and onto the ramp 2501, the ramp may flex slightly against the stylet 2610 but can maintain a shape, rather than deflect backward, to guide the guidewire outward toward the surrounding environment 222.

The guidewire lumen 308, like the stylet lumen 2510, can contain a supportive component. More particularly, as described above, the isolation tube 530 can be disposed in the guidewire lumen 308. The isolation tube 530 can provide a channel though which the guidewire passes, and can also support other components of the tissue treatment catheter 102. For example, the ultrasound transducer 208, which may be configured to emit acoustic energy, can be mounted on the isolation tube 530. The isolation tube 530 may also support the balloon 112, which as described above, can be mounted on the catheter shaft 202 and have an interior 310 in fluid communication with the fluid lumen 502. The ultrasound transducer 208 may be contained within the interior 310 of the balloon 112.

In an embodiment, the isolation tube 530 includes a notch 2620. The notch 2620 can be formed at a proximal end of the isolation tube 530 to allow a radially inward portion of the isolation tube 530 to be located proximally relative to a radially outward portion of the isolation tube 530. More particularly, the isolation tube 530 can have a proximal notch end 2622 and a distal notch end 2624, and the proximal notch end 2622 can be proximally located relative to the distal notch end 2624. The longitudinal offset between the notch ends allows the proximal notch end 2622 to be proximal to the port edge 2406 and the distal notch end 2624 to be distal to the port edge 2406. Accordingly, the guidewire can ride over the isolation tube 530 and be directed upward through the notch 2620 that is aligned with the guidewire port 220 into the surrounding environment 222.

The notch 2620 may be formed by removing an upper section of the isolation tube 530. When the upper section is removed, it can form a semi-circular wall section that is proximal to a circular wall section. In an embodiment, the semi-circular wall section can be located within the seam 2606. For example, the semi-circular wall section can be sandwiched between the ramp 2501 and the septum 2602. The sealant 2608 may therefore be dispensed along the interface between the isolation tube 530 and the port edge 2406 at the seam 2606.

The notched isolation tube 530 can provide support around the guidewire lumen 308 adjacent to the guidewire port 220. For example, the isolation tube 530 can be formed from a polyimide tube that is more rigid than the catheter shaft material. Accordingly, the isolation tube 530 can maintain rigidity around the guidewire port 220 to reduce a likelihood of buckling of the guidewire port area. Nonetheless, the isolation tube 530 may be less rigid than the stylet 2610 and, thus, the tissue treatment catheter 102 distal to the guidewire port 220 may be more flexible than the tissue treatment catheter 102 proximal to the guidewire port 220. The relative flexibility can allow the tissue treatment catheter 102 to have good pushability through the proximal section of the device, and to have good trackability through the distal section of the device, allowing the tissue treatment catheter 102 to navigate tortuous anatomies.

The tissue treatment catheter embodiments described above can allow access to a variety of tortuous anatomies throughout a patient anatomy. In an embodiment, the tissue treatment catheter 102 may be delivered through a radial access approach to vascular target sites. For example, radial access may be through a radial artery, a subclavian artery, and then into a downstream vessel having a sharp takeoff, such as a renal artery. Despite having a highly tortuous pathway with vascular arches, bends, or bifurcations, the support of the port brace and/or the hybrid stylet/isolation tube support described above can allow the tissue treatment catheter 102 to effectively navigate to the target vascular site without buckling. Notably, such tracking may be achieved without guide catheter support. More particularly, although delivery catheters typically require a guide catheter to support the catheter against buckling when being delivered through tortuous anatomies, the above embodiments can be tracked through such anatomies without external support. Such capability can be useful to physicians under many circumstances, including when tracking the tissue treatment catheter 102 into a pulmonary artery or vein. Such tracking must be performed without a guide catheter and can be achieved using the tissue treatment catheter 102 described herein. Similarly, some target anatomies may have exceptionally small profiles, e.g., inner diameters, which a guide catheter may not be able to access. For example, pancreatic vessels may be too small to permit both a delivery catheter and a guide catheter to enter. The tissue treatment catheter 102 described above can be used without a guide catheter and, thus, may access such anatomies that other delivery catheters would be unable to enter, unsupported, without buckling.

Referring to FIG. 27, a top view of a tissue treatment catheter is shown in accordance with an embodiment. The catheter shaft 202 is shown having the collapsed section 2402 longitudinally between the distal wall portion 2408 and the proximal wall portion 2410. The collapsed section 2402, which includes an indented ramp 2501 to guide the guidewire from the guidewire port 220 to the surrounding environment 222, can be bounded by the lateral boundaries 2506, the distal ramp end 2503, and the proximal ramp end 2504. As described above, the ramp 2501 is recessed into the page, as compared to the surrounding lateral boundaries 2506. Cross-sectional views along the section lines are now described for further understanding.

Referring to FIG. 28, a cross-sectional view, taken about line A-A of FIG. 27, of a distal wall portion of a tissue treatment catheter is shown in accordance with an embodiment. The distal wall portion 2408 of the catheter shaft 202 can include three or more lumens. The lumens may, for example, include the guidewire lumen 308, the cable lumen 1006, and one or more fluid lumens 502, e.g., a supply fluid lumen and a return fluid lumen. The lumens may be disposed about the longitudinal axis 2502, which can extend longitudinally through the septum 2602 of the catheter shaft 202. The septum 2602 can separate the shaft lumens.

The lumens may be configured to receive materials or objects. For example, the fluid lumens 502 can receive and circulate the inflation fluid 111. The isolation tube 530 may be disposed in the guidewire lumen 308 of the catheter shaft 202, and the guidewire lumen 308 can be defined by the isolation tube 530 because the guidewire can pass through the isolation tube 530. An electrical cable can be disposed in the cable lumen 1006 to deliver electrical signals to the ultrasound transducer 208. Notably, the outward facing surface of the outer shaft wall 402 in the distal wall portion 2408 can be circular and, thus, convex outward relative to the longitudinal axis 2502.

Referring to FIG. 29, a cross-sectional view, taken about line B-B of FIG. 27, of a guidewire port of a tissue treatment catheter is shown in accordance with an embodiment. The fluid lumen(s) 502 and the cable lumen 1006 can be shaped the same at a transverse cross-section at the guidewire port 220, as compared to the transverse cross-section at the distal wall portion 2408. The guidewire lumen 308, however, may be terminated by collapsing the outer shaft wall 402. For example, the inner wall surfaces can be squeezed and/or reflowed toward each other to define the seam 2606 at the port edge 2406. A notched section, e.g., a semi-circular tab, of the isolation tube 530 can be sandwiched between the inner walls 2604. The seam 2606 can have a u-shaped profile revealing the concave outward shape of the ramp 2501 relative to the longitudinal axis 2502. The ramp 2501 provides a smooth, grooved surface tapering upward to guide the guidewire toward the surrounding environment 222.

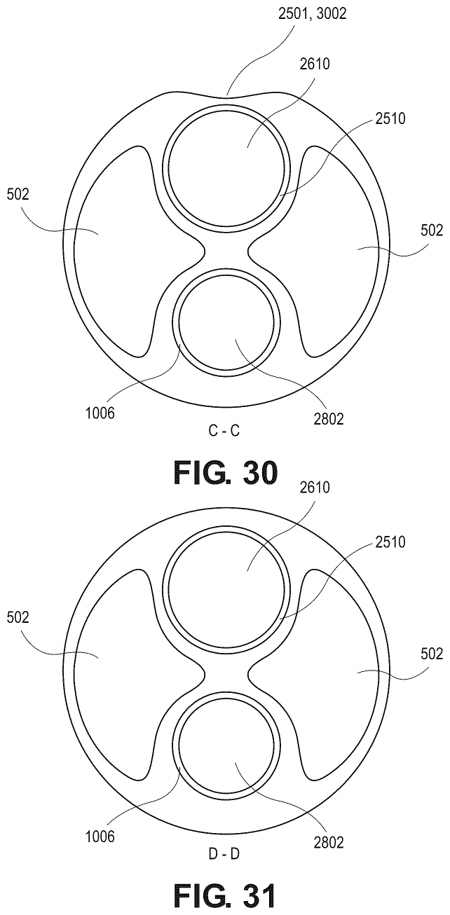

Referring to FIG. 30, a cross-sectional view, taken about line C-C of FIG. 27, of a ramp 2501 of a tissue treatment catheter is shown in accordance with an embodiment. Proximal to the guidewire port 220, the luminal profiles of the catheter shaft 202 can be identical to the luminal profiles distal to the guidewire port 220. More particularly, the fluid lumen(s) 502, the cable lumen 1006, and the stylet lumen 2510 near the proximal wall portion 2410 can have a same shape as the fluid lumen(s) 502, the cable lumen 1006, and the guidewire lumen 308 at the distal wall portion 2408. For example, the stylet lumen 2510, like the guidewire lumen 308, can be circular to receive the round stylet 2610. The outer shape of the catheter wall may, however, not be circular at the transverse plane location along the ramp 2501. More particularly, at a proximal end of the ramp 2501, the outer shaft wall 402 can have an indentation 3002. The indentation 3002 can be a concavity where the ramp 2501 is transitioning from the port edge 2406 to the proximal wall portion 2410. The concavity may be shallower than the concavity at the seam 2606, however, the outer surface of the catheter shaft 202 may nonetheless be non-circular.

Referring to FIG. 31, a cross-sectional view, taken about line D-D of FIG. 27, of a proximal wall portion of a tissue treatment catheter is shown in accordance with an embodiment. Proximal to the ramp 2501, the luminal profiles of the catheter shaft 202 can be identical to the luminal profiles distal to the guidewire port 220. More particularly, the fluid lumen(s) 502, the cable lumen 1006, and the stylet lumen 2510 in the proximal wall portion 2410 can have a same shape as the fluid lumen(s) 502, the cable lumen 1006, and the guidewire lumen 308 at the distal wall portion 2408. For example, the stylet lumen 2510, like the guidewire lumen 308, can be circular. The outer shape of the catheter wall may also be circular. More particularly, the proximal wall portion 2410 can be cylindrical.

Having described the tissue treatment catheter 102 having the collapsed section 2402, a method of manufacturing the tissue treatment catheter 102 is now described. It will be appreciated that the method is provided by way of example, and the operations described may be added to or subtracted from, including being performed in different orders, to manufacture the structures described above.

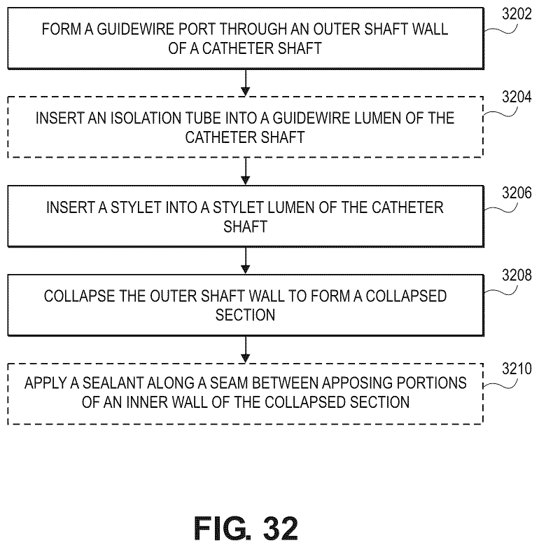

Referring to FIG. 32, a flowchart of a method of manufacturing a tissue treatment catheter is shown in accordance with an embodiment. At operation 3202, the guidewire port 220 is formed though the outer shaft wall 402 of the catheter shaft 202. More particularly, the outer shaft wall 402 can be extruded as a multi-lumen catheter having several lumens extending longitudinally from a proximal catheter end to a distal catheter end. The outer shaft wall 402 can extend around a single lumen, which has portions that will become the stylet lumen 2510 and the guidewire lumen 308. The stylet lumen 2510 portion is therefore coaxially aligned with the guidewire lumen 308 portion. When the guidewire port 220 is formed, e.g., by skiving, punching, drilling, or otherwise forming a hole through the cylindrical outer surface of the catheter shaft 202 into the single lumen, the guidewire port 220 can form a reference delineation between the stylet lumen 2510, proximal to the hole, and the guidewire lumen 308, distal to the hole. The guidewire port 220 includes the port edge 2406 extending around the hole. In the initial state, prior to reforming the catheter shaft 202 to fabricate the ramp 2501, the port edge 2406 can have a circular profile that is projected onto a cylindrical outer surface.

At operation 3204, optionally, the isolation tube 530 is inserted into the guidewire lumen 308 of the catheter shaft 202. The isolation tube 530 can have the notch 2620, and the isolation tube 530 can be inserted into the distal end of the guidewire lumen 308, in a proximal direction, until the proximal notch end 2622 passes proximal to the guidewire port 220. The isolation tube 530 inner surface, e.g., along the semi-circular wall around the notch 2620, can be visible through the guidewire port 220. The distal notch end 2624, however, may be aligned with or distal to the proximal end of the guidewire port 220.

At operation 3206, the stylet 2610 is inserted into the stylet lumen 2510. The stylet 2610 can be inserted through a proximal end of the stylet lumen 2510 and advanced distally until the distal stylet end 2612 is adjacent to the isolation tube 530. For example, the distal stylet end 2612 can abut the proximal notch end 2622. It will be understood that, when the collapsed section 2402 is formed, the distal stylet end 2612 can abut the collapsed section 2402 and, thus, the distal stylet end 2612 is inserted into the stylet lumen 2510 against the collapsed section 2402.

The lumens may be filled with corresponding mandrels. More particularly, mandrels can be inserted into the guidewire lumen 308, the fluid lumen(s) 502, and/or the cable lumen 1006. The mandrels can maintain a size and shape of the lumens when the collapsed section 2402 is formed, as described below. The mandrel located in the guidewire lumen 308 can pass through the guidewire port 220 and the isolation tube 530, arranged in the location through which the guidewire will eventually be able to pass.

At operation 3208, the outer shaft wall 402 is collapsed to form the collapsed section 2402. As described above, the collapsed section 2402 can taper outward from the port edge 2406 longitudinally between the stylet lumen 2510 and the guidewire lumen 308. Collapsing the outer shaft wall 402 can include a plastic reflowing process. A heat shrink tubing length can be placed over the catheter shaft 202 and aligned with the guidewire port 220. The assembly may be heated, e.g., by a heated air nozzle to melt the outer shaft wall 402. As the outer shaft wall 402 melts and the heat shrink tubing squeezes around the guidewire port 220, the mandrel in the guidewire lumen 308 can be squeezed downward, causing the port edge 2406 to reflow and to collapse against the septum 2602. The assembly may then be cooled, and the heat shrink tubing removed. The mandrel in the guidewire lumen 308 can be removed to expose the guidewire lumen 308 and reveal the ramp 2501 tapering into the guidewire port 220.

At operation 3210, optionally, the sealant 2608 may be applied between apposing portions of the inner wall 2604 of the collapsed section 2402 to seal the edge of the ramp 2501. For example, an ultraviolet-cured adhesive can be dispensed along the reflowed port edge 2406 and cured to fill any gaps between the inner walls 2604.

Additional method operations can be performed to complete the tissue treatment catheter 102. For example, the ultrasound transducer 208 can be mounted on the isolation tube 530, and the balloon 112 can be mounted on the catheter shaft 202 to place an interior 310 of the balloon 112 in fluid communication with the fluid lumen 502 of the catheter shaft 202. The method can therefore form a tissue treatment catheter 102 having a supported guidewire port 220 that is trackable to small and tortuous anatomies for use in neuromodulation.

Embodiments are described in the following enumerated examples.

Example 1. A tissue treatment catheter includes a catheter shaft, a balloon, and a port brace. The catheter shaft has a fluid lumen, a guidewire lumen, and a guidewire port extending through an outer shaft wall between the guidewire lumen and a surrounding environment. The balloon is mounted on the catheter shaft and has an interior in fluid communication with the fluid lumen. The port brace is disposed in the guidewire lumen. The port brace includes a proximal brace section in the guidewire lumen proximal to the guidewire port. The port brace includes a distal brace section in the guidewire lumen distal to the guidewire port. The proximal brace section is stiffer than the distal brace section.

Example 2. The tissue treatment catheter of example 1 further including a fluid lumen in the catheter shaft and a balloon mounted on the catheter shaft and having an interior in fluid communication with the fluid lumen.

Example 3. The tissue treatment catheter of example 2 further including an ultrasound transducer mounted on the catheter shaft and contained within the interior of the balloon. The ultrasound transducer is configured to emit acoustic energy.

Example 4. The tissue treatment catheter of example 2. The catheter shaft includes one or more of a second fluid lumen or a cable lumen.

Example 5. The tissue treatment catheter of example 1. A stiffness of the distal brace section decreases in a distal direction.

Example 6. The tissue treatment catheter of any of example 5. The distal brace section includes a brace tube having a slot.

Example 7. The tissue treatment catheter of any of example 6. The slot defines a helical wall and has a pitch that decreases in the distal direction.

Example 8. The tissue treatment catheter of example 5. A cross-sectional area of the distal brace section decreases in the distal direction.

Example 9. The tissue treatment catheter of example 1. The port brace includes a brace mandrel having a guidewire ramp located at a distal mandrel end.

Example 10. The tissue treatment catheter of example 9. The port brace includes a brace tube having a proximal collar mounted on the brace mandrel proximal to the guidewire ramp.

Example 11. The tissue treatment catheter of example 10. The brace tube includes a reinforcement bridge extending distally from the proximal collar to a distal tube segment to define a guidewire notch aligned with the guidewire port between the guidewire ramp and the distal tube segment.

Example 12. The tissue treatment catheter of example 11. The proximal brace section includes the brace mandrel and the proximal collar. The proximal brace section is stiffer than the reinforcement bridge.

Example 13. The tissue treatment catheter of example 1 further including an isolation tube extending through the guidewire lumen. The isolation tube includes a proximal tube end located distal to the guidewire port.

Example 14. The tissue treatment catheter of example 13. The proximal tube end is located proximal to a distal brace end of the port brace.

Example 15. The tissue treatment catheter of example 13. The proximal tube end is located distal to a distal brace end of the port brace.

Example 16. The tissue treatment catheter of example 13. The isolation tube includes an internal support wire.

Example 17. The tissue treatment catheter of example 1. The proximal brace section has a solid cross-sectional profile and the distal brace section has an annular cross-sectional profile.

Example 18. The tissue treatment catheter of example 17. The solid cross-sectional profile and the annular cross-sectional profile are concentrically disposed about a longitudinal axis of the guidewire lumen.

Example 19. A tissue treatment catheter includes a catheter shaft, a balloon, and a port brace. The catheter shaft has a fluid lumen, a cable lumen, a guidewire lumen, and a guidewire port extending through an outer shaft wall between the guidewire lumen and a surrounding environment. The balloon is mounted on the catheter shaft and has an interior in fluid communication with the fluid lumen. The port brace is located in the fluid lumen or the cable lumen. The port brace is aligned with the guidewire port. The port brace is stiffer than the outer shaft wall at the guidewire port.

Example 20. The tissue treatment catheter of example 19 further including an ultrasound transducer mounted on the catheter shaft and contained within the interior of the balloon. The ultrasound transducer is electrically connected to an electrical cable in the cable lumen.

Example 21. A tissue treatment catheter includes a catheter shaft, a balloon, and a port brace. The catheter shaft has a fluid lumen, a cable lumen, a guidewire lumen, and a guidewire port extending through an outer shaft wall between the guidewire lumen and a surrounding environment. The balloon is mounted on the catheter shaft and has an interior in fluid communication with the fluid lumen. The port brace is mounted on the outer shaft wall. The port brace is aligned with the guidewire port. The port brace is stiffer than the outer shaft wall at the guidewire port.

Example 22. The tissue treatment catheter of example 21 further including an ultrasound transducer mounted on the catheter shaft and contained within the interior of the balloon. The ultrasound transducer is electrically connected to an electrical cable in the cable lumen.

Example 23. A tissue treatment catheter including a catheter shaft and a port brace. The catheter shaft has a guidewire lumen and a guidewire port. The guidewire port extends through an outer shaft wall between the guidewire lumen and a surrounding environment. The port brace has a brace lumen coaxial with the guidewire lumen. The port brace includes a proximal brace section proximal to the guidewire port. The port brace includes a distal brace section distal to the guidewire port.

Example 24. The tissue treatment catheter of example 23. The proximal brace section is stiffer than the distal brace section.

Example 25. The tissue treatment catheter of example 23. The port brace includes a reinforcement bridge longitudinally aligned with the guidewire port. The reinforcement bridge includes a brace wall opposite of the guidewire port. The brace wall is solid.

Example 26. The tissue treatment catheter of example 23. The port brace includes a brace mandrel having a guidewire ramp located distal to a proximal notch edge of the guidewire notch.

Example 27. The tissue treatment catheter of example 23. An outer surface of the port brace is roughened.

Example 28. The tissue treatment catheter of example 27. An inner surface of the port brace defines the brace lumen. The outer surface of the port brace is rougher than the inner surface of the port brace.

Example 29. The tissue treatment catheter of example 23. The distal brace section includes a slot having a pitch. The pitch is constant.

Example 30. The tissue treatment catheter of example 23. The port brace includes a brace tube mounted on a brace mandrel. The brace tube includes a guidewire notch aligned with the guidewire port. The guidewire notch is defined by a notch edge. The tissue treatment catheter further includes a weld between the brace tube and the brace mandrel along the notch edge.

Example 31. The tissue treatment catheter of example 30. The brace tube includes a guidewire notch aligned with the guidewire port. A distal notch edge of the guidewire notch is distal to a distal port edge of the guidewire port.

Example 32. The tissue treatment catheter of example 23. The port brace includes a reinforcement bridge longitudinally aligned with the guidewire port. The reinforcement bridge transitions into the distal brace section at a chamfer.

Example 33. The tissue treatment catheter of example 23. The port brace includes a reinforcement bridge between the proximal brace section and the distal brace section. The proximal brace section has a tubular wall. The reinforcement bridge has a first arc-shaped wall. The distal brace section has a second arc-shaped wall.

Example 34. The tissue treatment catheter of example 33. The first arc-shaped wall has a larger arc angle than the second arc-shaped wall.

Example 35. A tissue treatment catheter. The tissue treatment catheter includes a catheter shaft having an outer shaft wall extending around a stylet lumen coaxially aligned with a guidewire lumen. The catheter shaft includes a port edge defining a guidewire port extending through the outer shaft wall into the guidewire lumen. The outer shaft wall has a collapsed section tapering outward from the port edge longitudinally between the stylet lumen and the guidewire lumen. The tissue treatment catheter includes a stylet disposed in the stylet lumen.

Example 36. The tissue treatment catheter of example 35. The collapsed section defines a ramp from the guidewire lumen to a surrounding environment. The ramp longitudinally separates the guidewire lumen from the stylet lumen.

Example 37. The tissue treatment catheter of example 35. The outer shaft wall includes a distal wall portion distal to the port edge and a proximal wall portion proximal to the port edge. The distal wall portion and the proximal wall portion are cylindrical. The collapsed section is non-cylindrical.

Example 38. The tissue treatment catheter of example 35. The collapsed section is concave outward relative to a longitudinal axis of the catheter shaft.

Example 39. The tissue treatment catheter of example 35. The tissue treatment catheter further includes an isolation tube disposed in the guidewire lumen.

Example 40. The tissue treatment catheter of example 39. The isolation tube includes a notch having a proximal notch end and a distal notch end. The proximal notch end is proximal to the port edge. The distal notch end is distal to the port edge.

Example 41. The tissue treatment catheter of example 39. The tissue treatment catheter includes an ultrasound transducer mounted on the isolation tube. The ultrasound transducer is configured to emit acoustic energy.

Example 42. The tissue treatment catheter of example 41. The catheter shaft includes a fluid lumen. The tissue treatment catheter further includes a balloon mounted on the catheter shaft and having an interior in fluid communication with the fluid lumen. The ultrasound transducer is contained within the interior of the balloon.

Example 43. The tissue treatment catheter of example 35. The collapsed section includes an inner wall having portions that appose each other along a seam.

Example 44. The tissue treatment catheter of example 43. The tissue treatment catheter includes a sealant along the seam.

Example 45. A method of manufacturing a tissue treatment catheter. The method includes forming a guidewire port through an outer shaft wall of a catheter shaft. The outer shaft wall extends around a stylet lumen coaxially aligned with a guidewire lumen. The guidewire port has a port edge. The method includes collapsing the outer shaft wall to form a collapsed section tapering outward from the port edge longitudinally between the stylet lumen and the guidewire lumen. The method includes inserting a stylet into the stylet lumen.

Example 46. The method of example 45. The stylet includes a distal stylet end. The distal stylet end is inserted into the stylet lumen against the collapsed section.

Example 47. The method of example 45. The method includes inserting an isolation tube into the guidewire lumen.

Example 48. The method of example 47. The method includes mounting an ultrasound transducer on the isolation tube. The ultrasound transducer is configured to emit acoustic energy.

Example 49. The method of example 48. The method includes mounting a balloon on the catheter shaft to place an interior of the balloon in fluid communication with a fluid lumen of the catheter shaft. The balloon contains the ultrasound transducer.

Example 50. The method of example 45. The method includes applying a sealant along a seam between apposing portions of an inner wall of the collapsed section.

In the foregoing specification, the invention has been described with reference to specific exemplary embodiments thereof. It will be evident that various modifications may be made thereto without departing from the broader spirit and scope of the invention as set forth in

What is claimed is:

1. A tissue treatment catheter, comprising:
a catheter shaft having a guidewire lumen and a guidewire port extending through an outer shaft wall between the guidewire lumen and a surrounding environment; and
a port brace disposed in the guidewire lumen, wherein the port brace includes a proximal brace section in the guidewire lumen proximal to the guidewire port, and a distal brace section in the guidewire lumen distal to the guidewire port, and wherein the proximal brace section is stiffer than the distal brace section.

2. A tissue treatment catheter of claim 1, further comprising a fluid lumen in the catheter shaft and a balloon mounted on the catheter shaft and having an interior in fluid communication with the fluid lumen.

3. The tissue treatment catheter of claim 2, further comprising an ultrasound transducer mounted on the catheter shaft and contained within the interior of the balloon, wherein the ultrasound transducer is configured to emit acoustic energy.

4. The tissue treatment catheter of claim 2, wherein the catheter shaft includes one or more of a second fluid lumen or a cable lumen.

5. The tissue treatment catheter of claim 1, wherein a stiffness of the distal brace section decreases in a distal direction.

6. The tissue treatment catheter of claim 5, wherein the distal brace section includes a brace tube having a slot.

7. The tissue treatment catheter of claim 6, wherein the slot defines a helical wall and has a pitch that decreases in the distal direction.

8. The tissue treatment catheter of claim 5, wherein a cross-sectional area of the distal brace section decreases in the distal direction.

9. The tissue treatment catheter of claim 1, wherein the port brace includes a brace mandrel having a guidewire ramp located at a distal mandrel end.

10. The tissue treatment catheter of claim 9, wherein the port brace includes a brace tube having a proximal collar mounted on the brace mandrel proximal to the guidewire ramp.

11. The tissue treatment catheter of claim 10, wherein the brace tube includes a reinforcement bridge extending distally from the proximal collar to a distal tube segment to define a guidewire notch aligned with the guidewire port between the guidewire ramp and the distal tube segment.

12. The tissue treatment catheter of claim 11, wherein the proximal brace section includes the brace mandrel and the proximal collar, and is stiffer than the reinforcement bridge.

13. The tissue treatment catheter of claim 1, further comprising an isolation tube extending through the guidewire lumen, wherein the isolation tube includes a proximal tube end located distal to the guidewire port.

14. The tissue treatment catheter of claim 13, wherein the proximal tube end is located proximal to a distal brace end of the port brace.

15. The tissue treatment catheter of claim 13, wherein the proximal tube end is located distal to a distal brace end of the port brace.

16. The tissue treatment catheter of claim 13, wherein the isolation tube includes an internal support wire.

17. The tissue treatment catheter of claim 1, wherein the proximal brace section has a solid cross-sectional profile and the distal brace section has an annular cross-sectional profile.

18. The tissue treatment catheter of claim 17, wherein the solid cross-sectional profile and the annular cross-sectional profile are concentrically disposed about a longitudinal axis of the guidewire lumen.

19. A tissue treatment catheter, comprising:
a catheter shaft having a fluid lumen, a cable lumen, a guidewire lumen, and a guidewire port extending through an outer shaft wall between the guidewire lumen and a surrounding environment;
a balloon mounted on the catheter shaft and having an interior in fluid communication with the fluid lumen; and
a port brace located in the fluid lumen or the cable lumen, wherein the port brace is aligned with the guidewire port, and wherein the port brace is stiffer than the outer shaft wall at the guidewire port.

20. The tissue treatment catheter of claim 19, further comprising an ultrasound transducer mounted on the catheter shaft and contained within the interior of the balloon, wherein the ultrasound transducer is electrically connected to an electrical cable in the cable lumen.

21. A tissue treatment catheter, comprising:
a catheter shaft having a fluid lumen, a cable lumen, a guidewire lumen, and a guidewire port extending through an outer shaft wall between the guidewire lumen and a surrounding environment;
a balloon mounted on the catheter shaft and having an interior in fluid communication with the fluid lumen; and
a port brace mounted on the outer shaft wall, wherein the port brace is aligned with the guidewire port, and wherein the port brace is stiffer than the outer shaft wall at the guidewire port.

22. The tissue treatment catheter of claim 21 further comprising an ultrasound transducer mounted on the catheter shaft and contained within the interior of the balloon, wherein the ultrasound transducer is electrically connected to an electrical cable in the cable lumen.

23. A tissue treatment catheter, comprising:
a catheter shaft having a guidewire lumen and a guidewire port extending through an outer shaft wall between the guidewire lumen and a surrounding environment; and
a port brace having a brace lumen coaxial with the guidewire lumen, wherein the port brace includes a proximal brace section proximal to the guidewire port, and a distal brace section distal to the guidewire port.

24. The tissue treatment catheter of claim 23, wherein the proximal brace section is stiffer than the distal brace section.

25. The tissue treatment catheter of claim 23, wherein the port brace includes a reinforcement bridge longitudinally aligned with the guidewire port, wherein the reinforcement bridge includes a brace wall opposite of the guidewire port, and wherein the brace wall is solid.

26. The tissue treatment catheter of claim 23, wherein the port brace includes a brace mandrel having a guidewire ramp located distal to a proximal notch edge of a guidewire notch.

27. The tissue treatment catheter of claim 23, wherein an outer surface of the port brace is roughened.

28. The tissue treatment catheter of claim 27, wherein an inner surface of the port brace defines the brace lumen, and wherein the outer surface of the port brace is rougher than the inner surface of the port brace.

29. The tissue treatment catheter of claim 23, wherein the distal brace section includes a slot having a pitch, and wherein the pitch is constant.

30. The tissue treatment catheter of claim 23, wherein the port brace includes a brace tube mounted on a brace mandrel, wherein the brace tube includes a guidewire notch aligned with the guidewire port, wherein the guidewire notch is defined by a notch edge, and further comprising a weld between the brace tube and the brace mandrel along the notch edge.

31. The tissue treatment catheter of claim 30, wherein the brace tube includes a guidewire notch aligned with the guidewire port, and wherein a distal notch edge of the guidewire notch is distal to a distal port edge of the guidewire port.

32. The tissue treatment catheter of claim 23, wherein the port brace includes a reinforcement bridge longitudinally aligned with the guidewire port, and wherein the reinforcement bridge transitions into the distal brace section at a bevel.

33. The tissue treatment catheter of claim 23, wherein the port brace includes a reinforcement bridge between the proximal brace section and the distal brace section, wherein the proximal brace section has a tubular wall, wherein the reinforcement bridge has a first arc-shaped wall, and wherein the distal brace section has a second arc-shaped wall.

34. The tissue treatment catheter of claim 33, wherein the first arc-shaped wall has a larger arc angle than the second arc-shaped wall.

35. A tissue treatment catheter, comprising:
a catheter shaft having an outer shaft wall extending around a stylet lumen coaxially aligned with a guidewire lumen, wherein the catheter shaft includes a port edge defining a guidewire port extending through the outer shaft wall into the guidewire lumen, and wherein the outer shaft wall has a collapsed section tapering outward from the port edge longitudinally between the stylet lumen and the guidewire lumen; and
a stylet disposed in the stylet lumen.

36. The tissue treatment catheter of claim 35, wherein the collapsed section defines a ramp from the guidewire lumen to a surrounding environment, and wherein the ramp longitudinally separates the guidewire lumen from the stylet lumen.

37. The tissue treatment catheter of claim 35, wherein the outer shaft wall includes a distal wall portion distal to the port edge and a proximal wall portion proximal to the port edge, wherein the distal wall portion and the proximal wall portion are cylindrical, and wherein the collapsed section is non-cylindrical.

38. The tissue treatment catheter of claim 35, wherein the collapsed section is concave outward relative to a longitudinal axis of the catheter shaft.

39. The tissue treatment catheter of claim 35 further comprising an isolation tube disposed in the guidewire lumen.

40. The tissue treatment catheter of claim 39, wherein the isolation tube includes a notch having a proximal notch end and a distal notch end, wherein the proximal notch end is proximal to the port edge, and wherein the distal notch end is distal to the port edge.

41. The tissue treatment catheter of claim 39 further comprising an ultrasound transducer mounted on the isolation tube, wherein the ultrasound transducer is configured to emit acoustic energy.

42. The tissue treatment catheter of claim 41, wherein the catheter shaft includes a fluid lumen, and further comprising a balloon mounted on the catheter shaft and having an interior in fluid communication with the fluid lumen, and wherein the ultrasound transducer is contained within the interior of the balloon.

43. The tissue treatment catheter of claim 35, wherein the collapsed section includes an inner wall having portions that appose each other along a seam.

44. The tissue treatment catheter of claim 43 further comprising a sealant along the seam.

45. A method of manufacturing a tissue treatment catheter, comprising:
forming a guidewire port through an outer shaft wall of a catheter shaft, wherein the outer shaft wall extends around a stylet lumen coaxially aligned with a guidewire lumen, and wherein the guidewire port has a port edge;
collapsing the outer shaft wall to form a collapsed section tapering outward from the port edge longitudinally between the stylet lumen and the guidewire lumen; and
inserting a stylet into the stylet lumen.

46. The method of claim 45, wherein the stylet includes a distal stylet end, and wherein the distal stylet end is inserted into the stylet lumen against the collapsed section.

47. The method of claim 45 further comprising inserting an isolation tube into the guidewire lumen.

48. The method of claim 47 further comprising mounting an ultrasound transducer on the isolation tube, wherein the ultrasound transducer is configured to emit acoustic energy.

49. The method of claim 48 further comprising mounting a balloon on the catheter shaft to place an interior of the balloon in fluid communication with a fluid lumen of the catheter shaft, wherein the balloon contains the ultrasound transducer.

50. The method of claim 45 further comprising applying a sealant along a seam between apposing portions of an inner wall of the collapsed section.
